(19) United States
(12) Patent Application Publication
(10) Pub. No.: US 2018/0042483 A1
(43) Pub. Date: Feb. 15, 2018

(54) SYSTEMS AND METHODS FOR HYPERSPECTRAL IMAGING

(71) Applicant: Massachusetts Institute of Technology, Cambridge, MA (US)

(72) Inventors: Neelkanth M. Bardhan, Boston, MA (US); Xiangnan Dang, Cambridge, MA (US); Jifa Qi, West Roxbury, MA (US); Angela M. Belcher, Lexington, MA (US)

(21) Appl. No.: 15/558,162

(22) PCT Filed: Mar. 7, 2016
(86) PCT No.: PCT/US2016/021171

§ 371 Date: Sep. 13, 2017

Related U.S. Application Data
(60) Provisional application No. 62/143,723, filed on Apr. 6, 2015.

(51) Int. Cl. A61B 5/00; G01N 21/64; G01J 3/32; G01J 3/44; G01J 3/28; G01N 33/22

(52) U.S. Cl. CPC A61B 5/0071; A61B 5/0091; G01J 3/2823; G01N 33/22; G01J 3/32; G01J 3/4406; G01N 21/64; A61B 2576/00; G01N 2021/6421; G01N 2201/12

(57) ABSTRACT

A method, and corresponding system, can include identifying a plurality of wavelength spectral band components in hyperspectral image data, the spectral band components corresponding to mutually distinct sources of image contrast. An intensity image corresponding to each respective spectral band component can be calculated, followed by combining the respective intensity images to form an inter-band image based on the respective, mutually distinct sources of image contrast for each spectral band component. Intensity images can be hyperspectral or hyperdiffuse images. Hyperdiffuse imaging can be performed for each spectral band component identified using hyperspectral measurements. Spectral position and spectral width images corresponding to each spectral band component can be calculated and used to determine depth of features inside a surface of the target. Diffuse width images can be calculated from hyperdiffuse image data and used to determine depth.

### RELATED APPLICATIONS

This application claims the benefit of U.S. Provisional Application No. 62/143,723, filed on Apr. 6, 2015. The entire teachings of the above application(s) are incorporated herein by reference.

### BACKGROUND

It is fairly well established that biomedical imaging is one of the pillars of comprehensive healthcare, forming an important component of clinical protocols for treatment of cancers and infectious diseases. Imaging is an integral part of clinical decision-making during screening, diagnosis, staging, therapy planning and guidance, treatment and real-time monitoring of patient response, because of its ability to provide morphological, structural, metabolic and functional information at various spatio-temporal scales of interest, while being a minimally invasive and highly targeted source of physiological evidence.

There are few main imaging modalities available clinically, classified according to the image contrast mechanism: X-ray (2-D film imaging and computed tomography (CT)), positron emission tomography (PET), single photon emission computed tomography (SPECT), magnetic resonance imaging (MM), ultrasound, intravital microscopy (confocal, multiphoton), and optical imaging.

In recent years, there has been much interest in the exploration of optical imaging in vivo, due to the development of near-infrared (NIR) fluorescent probes, effective targeting agents, and custom-built imagers. It is known that NIR light has the ability to penetrate human tissue more deeply than visible wavelengths, and some custom imaging instrumentation has been developed for the NIR-II wavelength region (900-1,400 nm). In particular, liquid nitrogen-cooled InGaAs CCD detectors, which have quantum efficiency 85-90% in the 900-1,700 nm wavelength range, have been developed.

### SUMMARY

There are significant challenges remaining in imaging in the NIR-II wavelength domain with InGaAs detectors. For example, there is only a limited selection of fluorescent probes that emit in the NIR-II range favorable for medical imaging. Further, it is still necessary to increase signal-to-noise (S/N) ratios for high-sensitivity deep-tissue imaging in the NIR-II range. Another challenge is that heterogeneous, turbid biological media cause diffuse light scattering. The diffuse light scattering results in a trade-off between depth and resolution.

Furthermore, despite the significant advances in both the imaging instrumentation and methods for image processing, most of the aformentioned imaging techniques suffer from poor sensitivity and/or resolution and/or depth of focus in the three spatial dimensions (3-D), which preclude their applicability in detecting small numbers of cells at the very early stages of disease. MRI, while offering good resolution and depth of penetration in tissue, requires large, expensive hardware, which makes it inaccessible to people in rural areas and in less affluent communities, and requires long acquisition times. CT and PET-CT, while offering very good penetration depth and contrast, expose the patient to ionizing radiation sources such as X-rays or other radioactive materials. The increasing exposure to radiation from the widespread clinical prevalence of CT scans has caused growing concern about the occurrence of radiation-induced cancer. Although ultrasound is a fairly cost-effective technique with a good resolution, it is hampered by poor penetration depth in tissue. Microscopy techniques based on fluorescence (e.g., confocal or multi-photon imaging) enable visualization of vascular-level or cellular-level mechanisms; however, they are not suitable for rapid diagnostics at the macroscopic scale nor deep penetration in tissue.

One example clinical challenge remaining is to be able to image biological features with sufficient sensitivity for detection at the cellular level (in a complex tissue environment at deep penetration), which could then be used to identify and treat small tumor masses before the angiogenic switch growth phase, which is below the threshold of detection of most current imaging technologies.

Methods and systems described herein can provide imaging with high resolution and high depth penetration, using optical wavelengths and without ionizing radiation. Imaging can be done even at the cellular level, enabling early disease detection, while also allowing imaging of larger areas such as whole organs or whole bodies. Diffuse imaging and hyperspectral imaging can be combined in some embodiments to further increase image contrast. In addition to biological targets, other targets such as hydrocarbons can also be imaged with embodiment systems and methods.

In one embodiment, a method, and corresponding system, can include identifying a plurality of wavelength spectral band components in hyperspectral image data, the spectral band components corresponding to mutually distinct sources of image contrast. The method can also include calculating respective intensity images corresponding to each respective spectral band component, followed by combining the respective intensity images to form an inter-band image based on the respective, mutually distinct sources of image contrast for each spectral band component.

Calculating the respective intensity images can include performing an intra-band pixel-wise analysis of one or more of the spectral band components. Combining the respective intensity images to form an inter-band image can include performing an inter-band pixel-wise analysis by dividing individual pixel values of one of the intensity images by corresponding individual pixel values of another of the intensity images. Performing the inter-band pixel-wise analysis can further includes dividing individual pixel values of more than one of the intensity images by corresponding individual pixel values of others of the respective intensity images, respectively, to form a plurality of inter-band images. The method can also include determining an inter-band image of greatest image contrast.

The respective intensity images can be respective diffuse intensity images, and the method can also include obtaining hyperdiffuse image data for each spectral band component in the hyperspectral image data. The method can include enhancing contrast for a respective diffuse intensity image based on a maximum radial distance max r calculated from a plurality of radial distances r, where r is a distance between a given pixel of the respective hyperdiffuse image data and a center pixel corresponding to an incident beam location identified in the respective hyperdiffuse image data. The method can further include enhancing contrast for each respective diffuse intensity image based on a median r or based on a principal component analysis (PCA) score r(PCA).

The method can also include calculating respective diffuse width images corresponding to respective spectral band components to provide depth information for features in the inter-band image, the depth being depth inside a surface of a target represented in the inter-band image. Obtaining hyperdiffuse image data for each spectral band component can include using respective optical bandpass filters corresponding to respective spectral band components. Obtaining the hyperspectral data and/or the hyperdiffuse image data can include using a forward imaging mode, with a target medium being in an optical path between a light source illuminating the target medium and a detector array configured to detect the hyperspectral and/or hyperdiffuse image, the inter-band image being an image of the target medium. As an alternative, a reflectance imaging mode can be used to obtain the data, with a detector array positioned to substantially avoid detection of light from a light source illuminating the target medium. An angular imaging mode can also be used to obtain the data, with a target medium being in an optical path between a light source illuminating the target medium, a detector positioned at an angle with respect to a path between the illuminating light source and the target in a range of about 0° to about 180°.

The respective intensity images can be respective spectral intensity images, and calculating the respective spectral intensity images can include using the hyperspectral image data as source data. Calculating each respective spectral intensity image can include calculating based on a wavelength of maximum intensity, a wavelength of median intensity, or a wavelength of highest principal component analysis score identified in the respective spectral band.

The method can also include ascertaining the mutually distinct sources of image contrast for the respective spectral band components based on spectral position images or spectral width images for the respective spectral bands. The target medium can be a three-dimensional (3-D) target medium and the inter-band image can be a 3-D image of a target medium, and the method can also include determining lateral, 2-D location of one or more features in the target medium and depth of the one or more features from a surface of the target medium. Determining depth of the one or more features can include determining depth, with anatomical co-registration, of a tumor, vasculature, immune cell, foreign material, exogenous contrast agent, target medium inhomogeneity. Identifying 2-D location of the one or more features can include applying a 2-D registration technique using an overlay of 2-D fluorescent images captured using a combined hyperspectral and diffuse NIR imaging system and bright-field projection images captured using a silicon camera for co-registration. Identifying 2-D location and depth of features in the target medium can include applying a 3-D registration technique using an overlay of 3-D fluorescent images captured using a combined hyperspectral and diffuse NIR imaging system, coupled with bright-field 3-D images, point clouds, or surface meshes captured using a 3-D scanner for anatomical co-registration. Determining depth can include basing depth on a combination of a spectral shift and a signal-to-background area. For HSC in FIG. 7B described hereinafter, for example, in the fourth band, combining information from 748ₖ (spectral intensity) and 748o (spectral width, indicating depth), or for HDC in FIG. 10, combining FIG. 10A (diffuse intensity) and FIG. 10B (diffuse width, indicating depth), give the 3-D information and produce 3-D images such as FIG. 13A. Determining depth can include determining a depth in a range from 0 cm to about 2 cm, in a range from about 2 cm to about 3.2 cm, in a range of about 3.2 cm to about 5 cm, or in a range of about 5 cm to about 9 cm.

The method can include performing an inter-band analysis to improve a signal-to-noise ratio in the inter-band image. The method can include obtaining the hyperspectral image data by collecting photons from a self-luminous target medium, which can include a bioluminescent organism expressing a luciferase or fluorescent protein. The method can also include obtaining the hyperspectral image data by illuminating a target medium with incident light, which can include using a light source having a wavelength between about 750 nm and about 1600 nm or between about 750 nm and about 1100 nm.

Illuminating the target medium with the incident light can include using incident light with a wavelength such that there is a wavelength separation between incident light, light inelastically scattered from the target medium, and at least one probe emission wavelength. Illuminating the target medium with the incident light can also include illuminating a probe introduced to the target medium, and identifying the plurality of spectral band components can include identifying a spectral band component corresponding to emission from the probe. Illuminating the probe can include illuminating a fluorescent probe, molecularly targeted reporter, or exogenous contrast agent, which can include a molecularly targeted fluorescent reporter, exogenous contrast agent, organometallic compound, doped metal complex, up-converting nanoparticle (UCNP), down-converting nanoparticle (DCNP), single-walled carbon nanotube (SWCNT), organic dye, or quantum dot (QD). Identifying the plurality of spectral band components can include identifying a spectral band component corresponding to an absorption or inelastic scattering of the incident light in the target medium or a target autofluorescence elicited by the incident light in the target medium.

Combining the respective intensity images to form the inter-band image can include forming an image of a cell, tissue, organ, tumor, whole body or fossil fuel. Combining to form the inter-band image can include forming an image with a resolution at a single-cell level. Identifying the plurality of spectral band components can include performing a principal component analysis on the hyperspectral image data to distinguish a probe emission from either an autofluorescence signal or a Raman scattering signal from a target medium. The inter-band image can be an image of a target body, and the method can also include obtaining the hyperspectral image data of the target body based on an anatomical core registration. The inter-band image can form a 3-D model of a target, and the method can further include overlaying a separate 3-D image from a 3-D scanner onto the 3-D model. A white light source can be used to register the inter-band image as part of the method.

The method can also include obtaining the hyperspectral image data without exogenous or endogenous labels, in a label-free manner. The spectral band components corresponding to mutually distinct sources of image contrast can result from heterogeneities inherent in a subject being imaged and represented in the hyperspectral image data or hyperdiffuse image data. The method can also include receiving the hyperspectral image date via a network connection or transmitting data representing the inter-band image via the network connection.

In another embodiment, an imaging system can include a detector configured to acquire hyperspectral image data for a target. The system can also include one or more processors configured to identify a plurality of wavelength spectral band components in the hyperspectral image data, the spectral band components corresponding to mutually distinct sources of image contrast, and the one or more processors being further configured to calculate respective intensity images corresponding to each respective spectral band component and to combine the respective intensity images to form an inter-band image based on the respective, mutually distinct sources of image contrast for each spectral band component.

In yet another embodiment, a method can include identifying a plurality of wavelength spectral band components in a hyperspectral image of a target, the spectral band components corresponding to mutually distinct sources of image contrast. The method can also include transforming each respective spectral band component to obtain a spectral position image and a spectral width image corresponding to each respective spectral band component. The method can also include calculating depth of one or more features inside a surface of the target based on the spectral position images and the spectral width images. Identifying the plurality of wavelength spectral band components can include identifying optical spectral band components.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a schematic block diagram illustrating use of an embodiment combined hyperspectral and hyperdiffuse optical imaging system, which may also be referred to as a system for Detection of Optically Luminescent Probes using Hyperspectral and Diffuse Imaging in Near-infrared (DOLPHIN) or a Combined Hyperspectral And Diffuse Near Infrared Imaging System (CHADNIS).

FIG. 2A is a flow diagram illustrating a method that can be used to obtain an inter-band image according to an embodiment.

FIG. 2B is a flow diagram illustrating various optional aspects of embodiment methods that can be used to obtain 2-D and 3-D images.

FIG. 2C is a schematic illustration of various sources of image contrast.

FIG. 2D is a graph illustrating tissue autofluorescence.

FIG. 3 is a flow diagram illustrating a method of determining depth of features inside a surface of a target.

FIG. 4A is a block diagram illustrating an imaging system that can be used as part of obtaining images according to embodiment methods illustrated in FIGS. 2A and 2B.

FIG. 4B is a schematic diagram illustrating a network in which embodiments of the system can be employed.

FIG. 5A illustrates a system for Detection of Optically Luminescent Probes using Hyperspectral and Diffuse Imaging in Near-infrared (NIR) (DOLPHIN) operating in hyperspectral imaging (HSI) mode.

FIG. 5B is a schematic illustration of the system shown in FIG. 5A.

FIG. 5C illustrates a system similar to that of FIG. 5A, except that it is configured to operate in hyperdiffuse imaging (HDI) mode.

FIG. 5D is a schematic illustration of the system shown in FIG. 5C.

FIGS. 5E-5G illustrate multi-level data processing for images captured in HSI mode, namely signal intensity as a function of XY position (FIG. 5E), level I processing of the data corresponding to the xy scanning (FIG. 5F), and level II processing of the image data corresponding to wavelength separation (FIG. 5G).

FIGS. 5H-5J are similar to FIGS. 5E-5G, except that data capture and processing illustrated are for HDI mode, with suitable optical band filters being used to collect the image data in FIG. 5H.

FIGS. 6A-6C are hyperspectral cube (HSC) representations of image data for an M-shaped feature, with FIG. 6A showing raw data, FIG. 6B showing projections on XY, XC, and YZ planes, and FIG. 6C showing an overlay stack of the emission spectrum of the M shaped feature at the two peak wavelengths in FIG. 6B.

FIGS. 7A-7C illustrate principal component analysis (PCA) applied to hyperspectral cube data obtained for an MIT-shaped feature located at a depth of 20 mm below breast tissue Phantom. FIG. 7A shows principal component values as a function of wavelength, contribution of principal components as a percentage of the first component, and mean intensity as a function of wavelength. FIG. 7B illustrates pixel-wise analysis of the HSC data, and FIG. 7C illustrates band division processing analysis.

FIG. 8A shows HSI data sets as a function of varying depths in tissue phantom from 0 to 40 mm.

FIG. 8B shows HSI data sets as a function of varying tissue properties.

FIGS. 9A-9C show imaging data in the form of a hyperdiffuse cube (HDC), in three different visualizations. FIG. 9A shows the raw HDC data, with the Z-axis corresponding to the radial distance r (scattering radius) from the center position of an incident beam illumination.

defined by the central spot of the laser illumination, FIG. 9B shows a projection on the XY plane, as well as along the radial dimension (XZ and YZ planes) of the “M”-shaped feature being imaged, and FIG. 9C shows the same projection as FIG. 9B with a transparency mask added with an opacity function proportional to the intensity of the colormap at each pixel.

FIGS. 10A-10C illustrate principal component analysis (PCA) applied to HDC data obtained for a “MIT”-shaped figure located at a depth of 20 mm below breast tissue Phantom shown in FIGS. 9A-9C. FIG. 10A shows PCA coefficient and mean intensity curves, FIG. 10B shows diffuse intensity images, and FIG. 10C shows diffuse with images.

FIGS. 11A-11B show HDI data sets as a function of varying depths in tissue phantom and varying tissue properties, respectively.

FIG. 11C is a graph illustrating variations of the transport scattering effect as a function of depth.

FIG. 11D is a bar chart illustrating variation of the transport scattering effect as a function of tissue environment.

FIG. 12 illustrates spectral intensity (SI), diffuse intensity (DI), and bright-field image overlays for hyperspectral and hyperdiffuse imaging in the NIR-II optical imaging range.

FIG. 13A shows a three-dimensional (3D) overlay of the diffuse intensity image shown in FIG. 10B with the scattering radius obtained from the diffuse width image shown in FIG. 10C. The overlay can be used for Z-depth estimation from analysis of HDC data.

FIG. 13B shows and XY planar projection of the image in FIG. 13A, constituting a two-dimensional (2D) fluorescent image.

FIG. 14A is a schematic diagram illustrating an embodiment system configured to analyze crude oil.

FIG. 14B is a graph showing known optical densities for various crude oil components that can be exploited for analysis using embodiment methods.

FIGS. 14C-14E are graphical images showing hyperspectral imaging data obtained using embodiment methods with various crude oil sample conditions.

FIGS. 15A-15J are a series of graphs illustrating measured effects of thickness and tissue type on the spectral and scattering properties identified by DOLPHIN.

FIGS. 16A-16M are graphs illustrating derivation of depth of signal and effective attenuation coefficient of tissues from fitting the results of tissue or animal penetration by DOLPHIN.

FIGS. 17A-17L are constructed graphical images illustrating fluorescence 3-D reconstruction of animal imaging.

FIGS. 18A-18E are constructed graphical images illustrating results of label-free scanning of a mouse.

### DETAILED DESCRIPTION

A description of example embodiments of the invention follows.

It is fairly well established that biomedical imaging is one of the pillars of comprehensive healthcare, forming an important component of clinical protocols for treatment of cancers and infectious diseases. Imaging is an integral part of clinical decision-making during screening, diagnosis, staging, therapy planning and guidance, treatment and real-time monitoring of patient response, because of its ability to provide morphological, structural, metabolic and functional information at various spatio-temporal scales of interest, while being a minimally invasive and highly targeted source of physiological evidence. The main clinical challenge, however, is to be able to image biological features with sufficient sensitivity for detection at the cellular level (in a complex tissue environment at deep penetration), which could then be used to identify and treat small tumor masses before the angiogenic switch growth phase, which is below the threshold of detection of most current imaging technologies.

There are few main imaging modalities available clinically, classified according to the image contrast mechanism: X-ray (2-D film imaging and computed tomography, CT), positron emission tomography (PET), single photon emission computed tomography (SPECT), magnetic resonance imaging (MM), ultrasound, intravital microscopy (confocal, multiphoton), optical imaging, or some combination of these. Despite the significant advances in both the imaging instrumentation and methods for image processing, most of the aforementioned techniques suffer from poor sensitivity and/or resolution and/or depth of focus in the three spatial dimensions (3-D), which preclude their applicability in detecting small numbers of cells at the very early stages of disease. MM, while offering good resolution and depth of penetration in tissue, requires large, expensive hardware, which makes it inaccessible to people in rural areas and in less affluent communities, and requires long acquisition times. CT and PET-CT, while offering very good penetration depth and contrast, expose the patient to ionizing radiation sources such as X-rays or other radioactive materials. The increasing exposure to radiation from the widespread clinical prevalence of CT scans has caused growing concern about the occurrence of radiation-induced cancer. Although ultrasound is a fairly cost-effective technique with a good resolution, it is hampered by poor penetration depth in tissue. Microscopy techniques based on fluorescence, e.g. confocal or multi-photon imaging, enable visualization of vascular-level or cellular-level mechanisms; however they are not suited for rapid diagnostics at the macroscopic scale and deep penetration. The most promising technique for high-resolution deep-tissue whole body imaging, using relatively safe molecular probes and excitation sources, at a reasonably low cost, appears to be optical imaging.

In recent years, there has been tremendous interest in the exploration of optical imaging in vivo, due to the development of near-infrared fluorescent probes, effective targeting agents, and custom-built imagers. The first driving force was the transition from visible wavelength emitters such as small-molecule organic dyes, fluorescently expressed proteins or quantum dots, to near-infrared fluorophores. This was motivated by the observation that near-infrared (NIR) light can travel through biological tissue more effectively, with reduced scattering and absorption at visible wavelengths, as well as improved spectral separation of probe emission from excitation and autofluorescence. NIR light has been claimed theoretically predicted to penetrate through ˜10 cm of human tissue, with simulations suggesting a signal-to-noise (S/N) improvement of over 100-fold by imaging in the second near-infrared window (NIR-II: 900-1,400 nm) compared to the first window (NIR-I: 650-900 nm). The second development was the wider availability of more effective functionalization and targeting agents for optical imaging.

A third factor motivating optical imaging in vivo, perhaps the most import factor, is the recent work on developing imaging instrumentation in the NIR-II regime. Commercial whole-animal imagers such as the Xenogen IVIS® Spectrum by Caliper Life Sciences are optimized for imaging in the visible, and to a certain extent in the NIR-I, due to their silicon CCD detectors, which have sharp fall-off in the responsivity beyond NIR-I. This has necessitated building custom imagers using liquid nitrogen-cooled InGaAs CCD detectors, which have quantum efficiency ˜85-90% in the 900-1,700 nm range.

However, there are three significant challenges to imaging in the NIR-II wavelength domain with InGaAs detectors. A first challenge is the limited selection of fluorescent probes which emit in the NIR-II range favorable for medical imaging. The available options include long-wavelength organic dyes, inorganic quantum dots (QDs) such as PbS or PbSe, single-walled semiconducting carbon nanotubes (SWNTs), and more recently, a class of lanthanide-doped fluoride nanoparticles emitting in up- or down-conversion mode (UCNPs). Among these options, organic dyes are less attractive due to their tendency to photobleach at stronger irradiances causing decrease in signal intensity over time, and QDs show high toxicity in vitro with concerns underscoring their potential for high in vivo toxicity, pending more substantive data proving otherwise. Organic dyes and QDs are also disadvantageous due to the relatively small spectral separation between excitation and emission, which makes distinguishing the probe signal from excitation or autofluorescence more difficult using idealized optical band-pass filters.

Previous work has demonstrated the application of functionalized, targeted SWNTs for whole animal in vivo imaging of cancers and bacterial infections, with large Stokes' shift, insensitivity to photobleaching, and with no apparent toxicity effects. One limitation of using SWNTs is their relatively high aspect ratio up to ˜1,000:1, which results in poor circulation characteristics upon intravenous injection, as evidenced by their relatively short half-lives˜few hours in blood, with a large fraction of the probe being captured by the macrophages of the liver and spleen and consequently relatively low probe uptake at the site of interest.

In contrast to SWNTs, UCNPs seem to offer the best possible balance of desirable fluorophore characteristics: (a) ability to be synthesized reproducibly in a very narrow size distribution, at ˜5-100 nm sizes, with suitable functionalization for targeted bioimaging applications with low non-specific binding and long circulation half-lives, (b) wavelength-tunable photoluminescence, with sharp emission in the NIR-II range by precise control of doping element and concentrations, and the photoluminescence wavelength mainly depends on the doping element, rather than the concentrations of doping or sizes of particle, (c) large Stokes' (down-conversion) or anti-Stokes' (up-conversion) shift, allowing better signal separation from the excitation source and tissue autofluorescence, (d) ability to be excited at high irradiances in the NIR regime, typically at 980 nm, due to relative insensitivity to photobleaching, with low absorption of the excitation wavelength by biological tissues minimizing potential for tissue damage, and (e) no apparent toxicity effects observed either in vitro or in vivo, in small pilot studies. Although there have been several cases of the application of UCNPs to in vivo imaging, the maximum reported depth of penetration is ˜3.2 cm in pork tissue using UCNPs, which is comparable to the value ˜2.5 cm in breast tissue phantom using SWNTs previously demonstrated. Both of these previously achieved depth range are significantly lower than the theoretical potential for deep-tissue optical imaging up to 10 cm.

A second challenge to imaging in the NIR-II wavelength domain with InGaAs detectors is to maximize the S/N ratio for high-sensitivity deep-tissue imaging. While 16-bit silicon CCDs can easily achieve baseline noise levels ˜1-10 (on a scale of 1 to 216=65,536), similar 16-bit InGaAs CCDs have baseline noise ˜100-1000 even when cooled to 173 K. This implies that for InGaAs detectors, the maximum theoretically achievable S/N is only ˜65, compared to S/N ˜6,500 for Si detectors. To circumvent this issue, it is important to keep other sources of background noise to a minimum, such as tissue autofluorescence. Biological tissues containing lipids scatter inelastically with strong Raman shifts 3,000, 2,800-3,000, 1,440 and 1,300 cm⁻¹, corresponding respectively to the unsaturated=C—H bond stretch, the saturated —CH₂ asymmetric and symmetric stretches, —CH₂ bend and —CH₂ twist vibrations. Therefore, for a 980 nm excitation source, it is beneficial if the probe emission is not centered close to ˜1,388 nm or around ˜1,135 nm, for mitigating the background. UCNPs such as NaYF₄ co-doped with Yb and Er are well-suited for this criterion, with a peak emission at 1,560 nm. Another workaround is to implement a technique known as hyperspectral imaging, which collects spectral information for each pixel of a 2-dimensional pixel array. The generated dataset known as the hyperspectral cube/(x, y, λ), where x and y are spatial coordinates and λ is the wavelength, enables probing the interactions of light with physiological features more completely, and thereby capturing subtle spectral differences arising from changes in pathology. While there have been some applications of hyperspectral imaging for clinical diagnostics, they have mostly utilized the visible or NIR-I wavelengths due to more well-established instrumentation. The available literature on NIR-II hyperspectral imaging has been limited to either surface-level visualization or as an intraoperative imaging tool, with most systems implemented in reflectance mode imaging, with apparently no whole-body deep imaging systems available. HSI resolves this challenge by providing information in frequency domain, not only allowing novel type of investigation, but also improving confidence in results.

A third challenge to imaging in the NIR-II wavelength domain with InGaAs detectors is diffuse light scattering by heterogeneous turbid biological media. Diffuse light scattering effectively imposes a trade-off between depth and resolution. Describing common method: DOT. Similar to HSI, HDI can be performed to resolve this challenge by addressing diffuse light scattering. This not only excludes the emission scattering in the processed results, but also presents pixel-wise diffuse scattering information for contrast imaging.

According to embodiments described herein, transilluminating optical imaging can be performed at depths of up to 9 cm in biological tissues, with high sensitivity to detect features. In some circumstances, resolution at single-cell level (tens of μm) can be achieved. Some embodiments described herein include a hyperspectral and diffuse imaging system operating at 900-1700 nm wavelengths. Embodiments have the capability to distinguish the optical signatures of a primary pump laser, background, tissue autofluorescence, and reporter fluorescence, as well as the diffuse scattering effect of the fluorescence signal upon transport through heterogeneous turbid optical media. A combination of strategies to acquire and analyze data can involve (a) innovative hardware design comprising 2-D spatial scanning coupled with hyperspectral imaging in transmission mode to improve S/N through intra-band and inter-band analyses, and (b) new image processing techniques leveraging the rich information obtained from hyperspectral cube and hyperdiffuse cube for depth- and environment-resolved imaging, and (c) rational materials selection based on NIR-II emitting UCNPs. Using such a combination of strategies, light-probe-physiological interactions can be investigated at various hierarchical scales of interest (whole body/organ or tissue/tumor microenvironment/cellular level).

To correlate experimental observations with the transport scattering phenomena, Monte Carlo simulations covering a palette of tissue types can be performed, with varying 3-D structures approximating real organs, at realistic depths ranging from 0 to 5 cm. High-resolution depth- and environment-resolved data can be reconstructed to obtain 3-D anatomical information from 2-D scans, thereby obviating the need for expensive, 360° tomographic hardware. Embodiment systems and methods can enable new possibilities for clinical translation of NIR-II imaging as a viable platform for theranostic technology, for early diagnostics, for real-time surgical assistance tools, and for monitoring patient response to therapies.

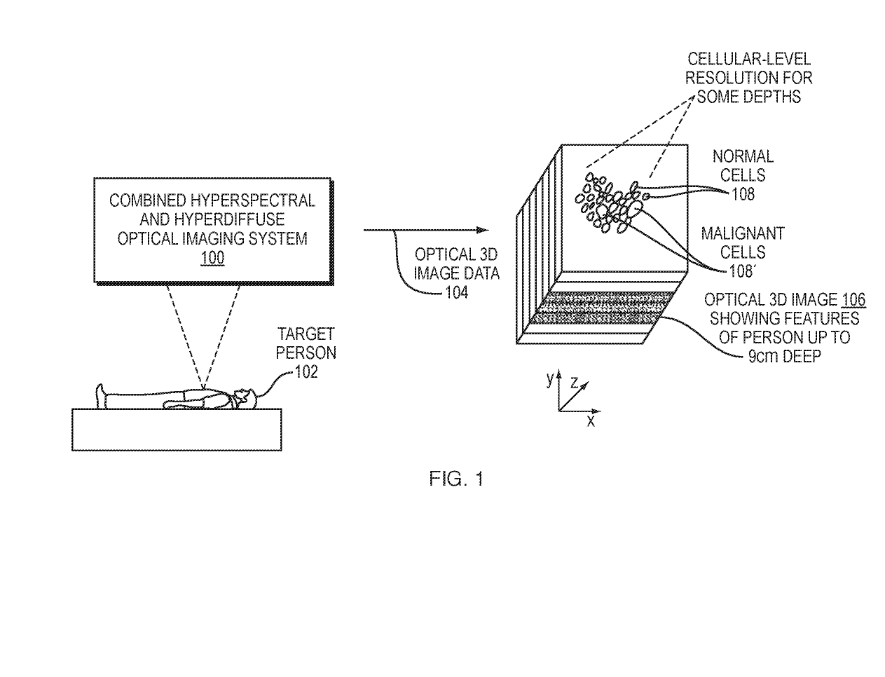

FIG. 1 is a schematic block diagram illustrating use of an embodiment combined hyperspectral and hyperdiffuse optical imaging system 100. The system 100 can be used to image a target person 102 or portion thereof. The imaging system 100 provides 3-D image data 104 based on optical wavelengths detected. The data can be processed according to embodiment methods to present a 3-D image 106 showing features of the target person 102. The features thus shown in the image 106 can be located at depths up to 9 cm below the surface (skin) of the person 102. Furthermore, for some depths and system configurations, cellular-level resolution can be obtained. For example, normal cells 108 can be distinguished from malignant cells 108′.

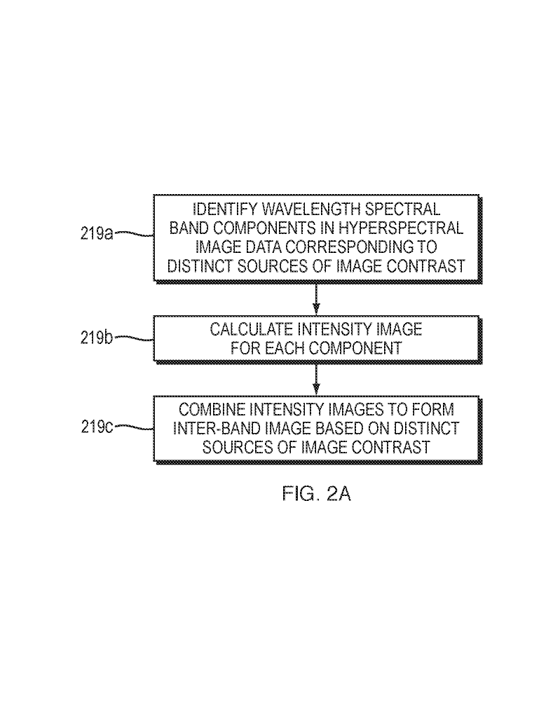

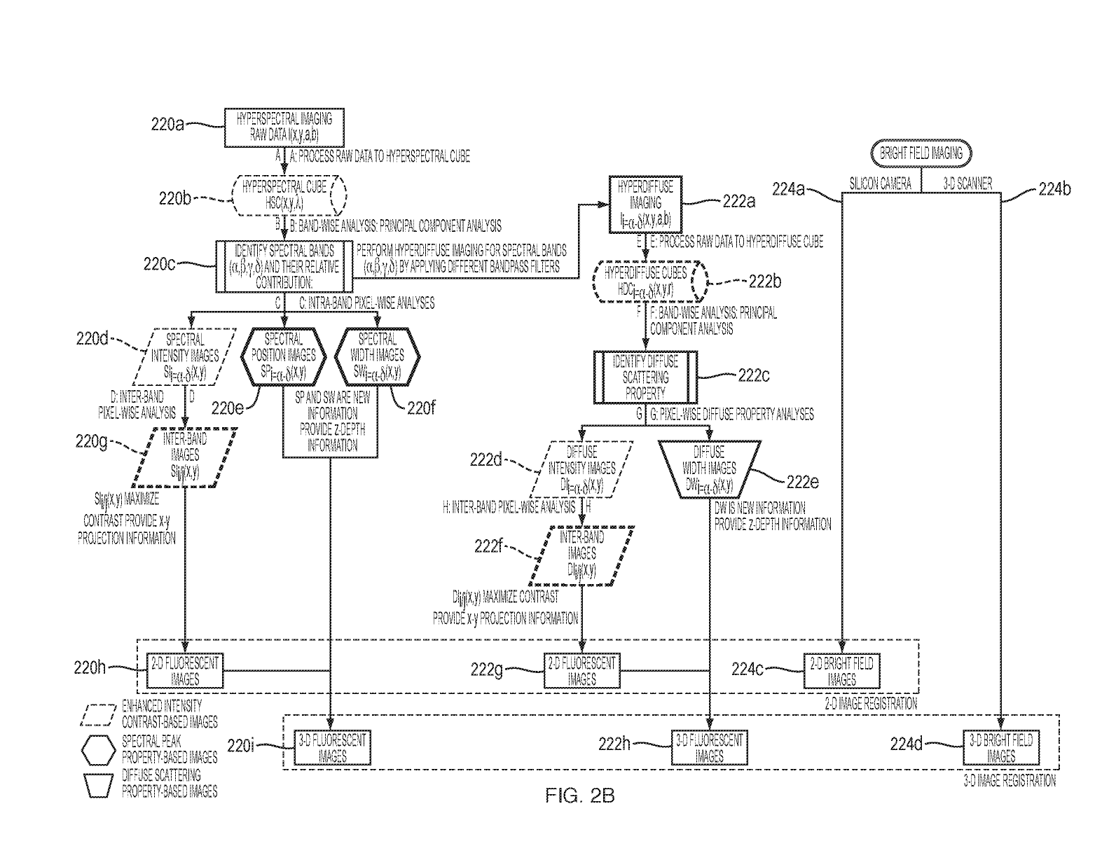

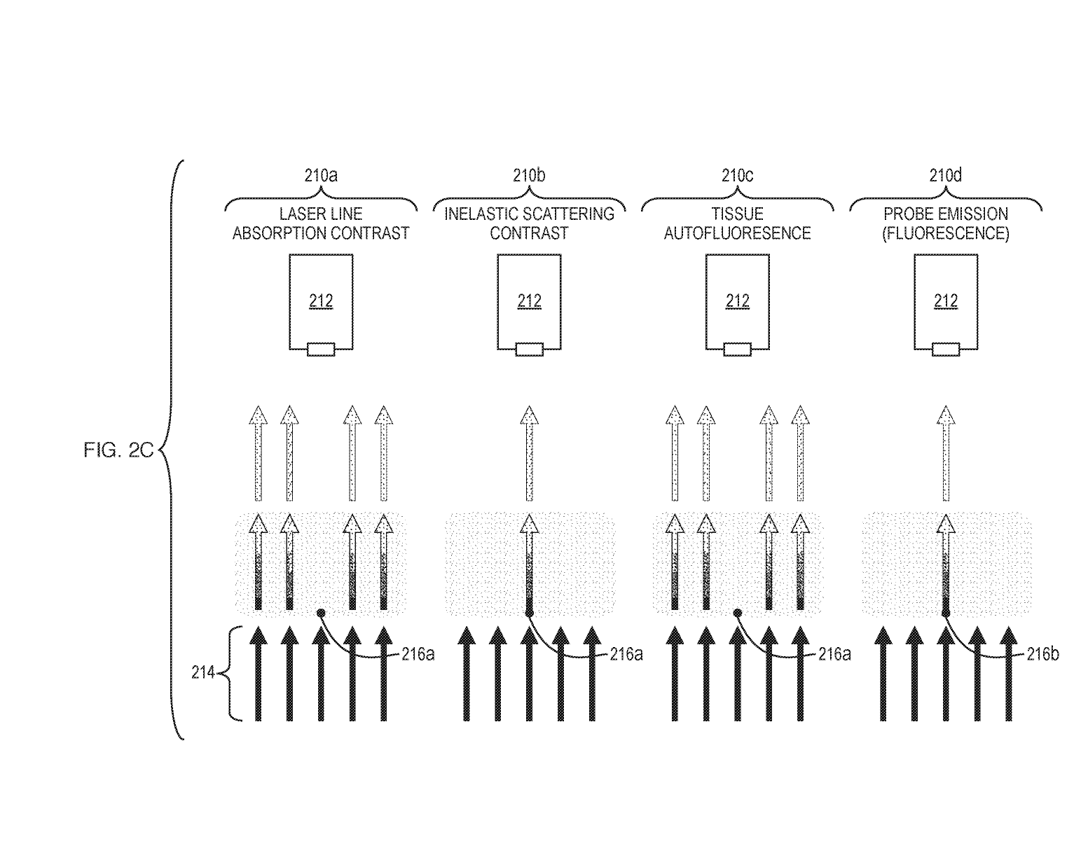

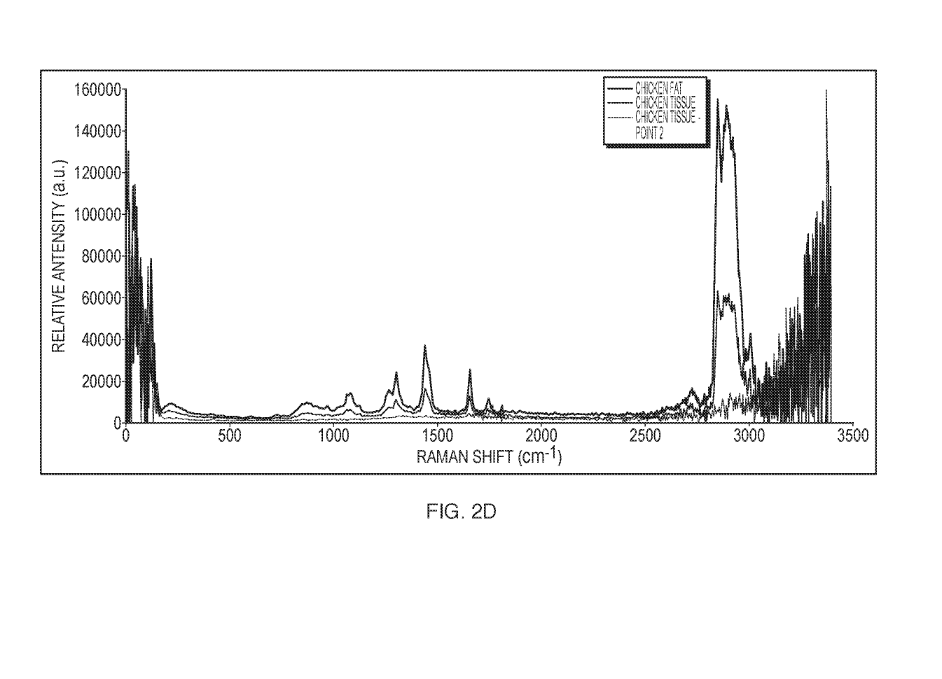

FIG. 2A is a flow diagram illustrating a method that can be used to obtain an inter-band image according to an embodiment. At 219a, a plurality of wavelength spectral band components in hyperpectral image data are identified, where the spectral band components correspond to mutually distinct sources of image contrast. At 219b a respective intensity image is calculated corresponding to each respective spectral band component. At. 219c, the respective intensity images are combined to form an inter-band image based on the respective, mutually distinct sources of image contrast for each spectral band component. In some embodiments, the intensity images are hyperspectral intensity images, while in other embodiments, the intensity images are diffuse intensity images obtained by performing hyperdiffuse imaging for each respective spectral band component identified in the hyperspectral image data.

FIG. 2B is a flow diagram illustrating various optional aspects of embodiment methods that can be used to obtain 2-D and 3-D images. Elements of the procedure illustrated in FIG. 2B are summarized in the following paragraphs for convenience, and various elements of the procedure are further described elsewhere in this application.

For the case of hyperspectral imaging (HSI), the directly measured results (raw data)I(x, y, a, b) at 100×100 positions (x, y) comprised of 320×256 intensity pixels (a, b) can be transformed to a hyperspectral cube (HSC) of 320 spectral bands, HSC(x, y, λ), where λ is the wavelength.

At 220a, raw hyperspectral imaging data are obtained. At A, the raw data are processed to form a hyperspectral cube at 220b according the following equation:

I(x,y,a,b)→I(x,y,λ,b)→I(x,y,λ):HSC(x,y,λ) A:

At B, a band-wise principal component analysis (PCA) is performed, and at 220c, wavelength spectral bands and their relative contribution are identified from the PCA, according to the following equation:

[Coeff(λ),Score(x,y),Explained,μ(λ)]=PCS(HSC(x,y,λ)) B:

with the functional domains for the four parameters defined as Coeff(λ, rank=1-4); Score(x, y, rank=1-4); Explained(rank=1-4); and μ(λ). The first parameter, Coeff, contains information describing the transformation of principal components from spectral bands. Coeff of the first 4 principal components (ordered by the relative contribution from each component to the HSC) are plotted to help identify the most pronounced spectral bands. Four bands have typically been identified based on PCA and the light-probe-tissue interaction, namely α-band: laser line (absorption contrast), β-band: probe emission I (1100 nm), γ-band: tissue autofluorescence and/or Raman scattering, and the δ-band: probe emission II (1550 nm). These are example bands that can constitute a plurality of wavelength spectral band components from the hyperspectral image data corresponding to mutually distinct sources of image contrast, as further illustrated in FIGS. 2C-2F.

The second parameter, Score, contains the linear combination processed image from each principal component listed in order of contribution. Most information has typically been found to be contained in the first three components, while the rest are dominated by noise. The third parameter, Explained, describes the contribution from each principal component to the measured results, HSC(x, y, λ). Depending on the complexity of the tissue sample/probe combination, up to 4 principal components contribute to the original HSC to some extent. Finally, the fourth parameter μ is the averaged intensity from each spectral frame, which also serves as an indicator for important bands (more evident for data with high SNR).

At C, various intra-band pixel-wise analyses are performed according to the equations below. Namely, at 220d, spectral intensity (SI) images are calculated, in this case using the hyperspectral image data as the source data. As illustrated further hereinafter, hyperdiffuse image data can be used to obtain diffuse intensity images, with the hyperdiffuse image data as the source data. At 220e, spectral position (SP) images are calculated, and at 220f, spectral width (SW) images are calculated.

### C:

$${SI}_{i = α \vert - δ} ( x , y ) = {max}_{λ} ( {HSC}_{i = α - δ} ( x , y , λ ( i ) ) ) OR$$

$${SI}_{i = α - δ} ( x , y ) = {median }_{λ} ( {HSC}_{i = α - δ} ( x , y , λ ( i ) ) ) OR$$

$${SI}_{i = α - δ} ( x , y ) = {PCA Score }_{λ} ( {HSC}_{i = α - δ} ( x , y , λ ( i ) ) )$$

$${SP}_{i = α - δ} ( x , y ) = {Peak Position }_{λ} ( {HSC}_{i = α - δ} ( x , y , λ ( i ) ) )$$

$${SW}_{i = α - δ} ( x , y ) = {Peak Width }_{λ} ( {HSC}_{i = α - δ} ( x , y , λ ( i ) ) )$$

where i denotes the i^{th} spectral band (α-δ). Intra-band analysis is performed on HSCᵢ to achieve pixel information for each band of Spectral Intensity, Position, and Width: denoted as SIᵢ, SPᵢ, and SWᵢ. The respective sources of image contrast, if not already known, can be ascertained based on the spectral position images or spectral width images for the respective spectral bands.

At D, based upon the SI images, an inter-band pixel-wise analysis can be performed to calculate various inter-band images at 220g, as further described hereinafter.

### D:

$${SI}_{i / j} ( x , y ) = \frac{{SI}_{i = α - δ} ( x , y )}{{SI}_{j = α - δ , j \neq i} ( x , y )}$$

is utilized to characterize and maximize the image contrast based on the knowledge of the origin of contrast of each spectral region. Respective intensity images are thus combined to form inter-band images based on the respective, mutually distinct sources of image contrast represented in the various spectral band components. Alternatively, as shown hereinafter, diffuse intensity images obtained from hyperdiffuse imaging can be combined to form the inter-band images based on the mutually distinct sources of image contrast in a similar way using a similar inter-band pixel-wise analysis, particularly dividing pixel values of one band image by those of other band images to form various inter-band images

A given inter-band image S_{i/j} can be selected to maximize image contrast to produce a high-contrast 2-D fluorescent image at 220h. The SP and SW images at 220e and 220f, respectively, can be used in conjunction with the 2-D fluorescent images at 220h to obtain 3-D fluorescent images at 220i, as further described hereinafter.

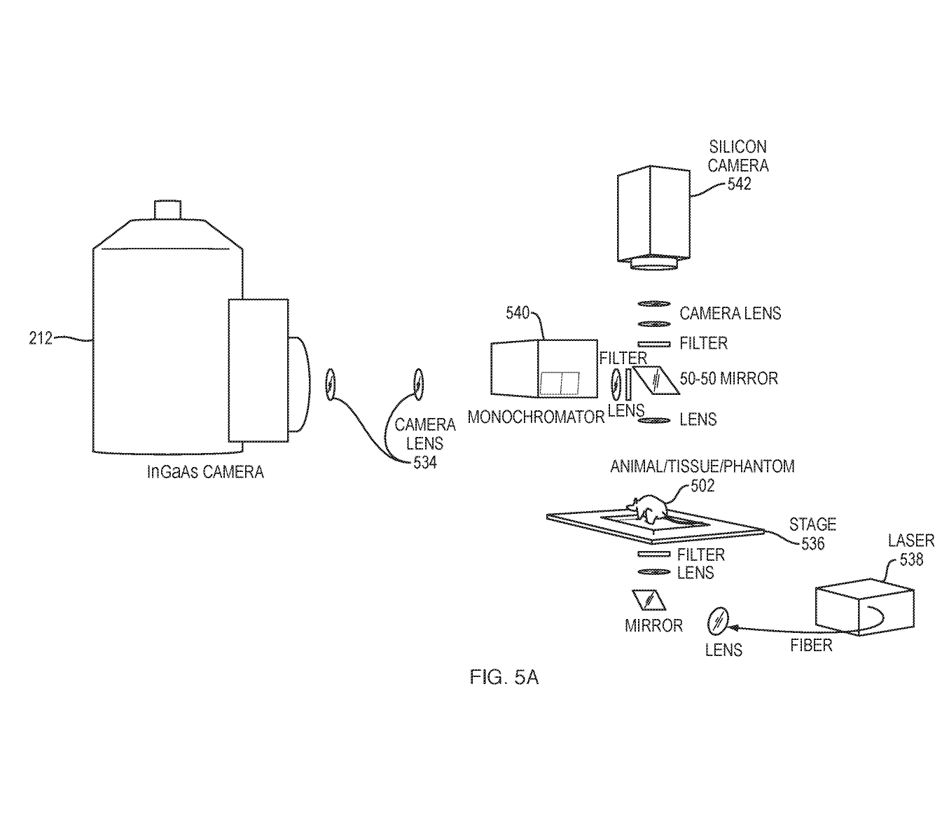

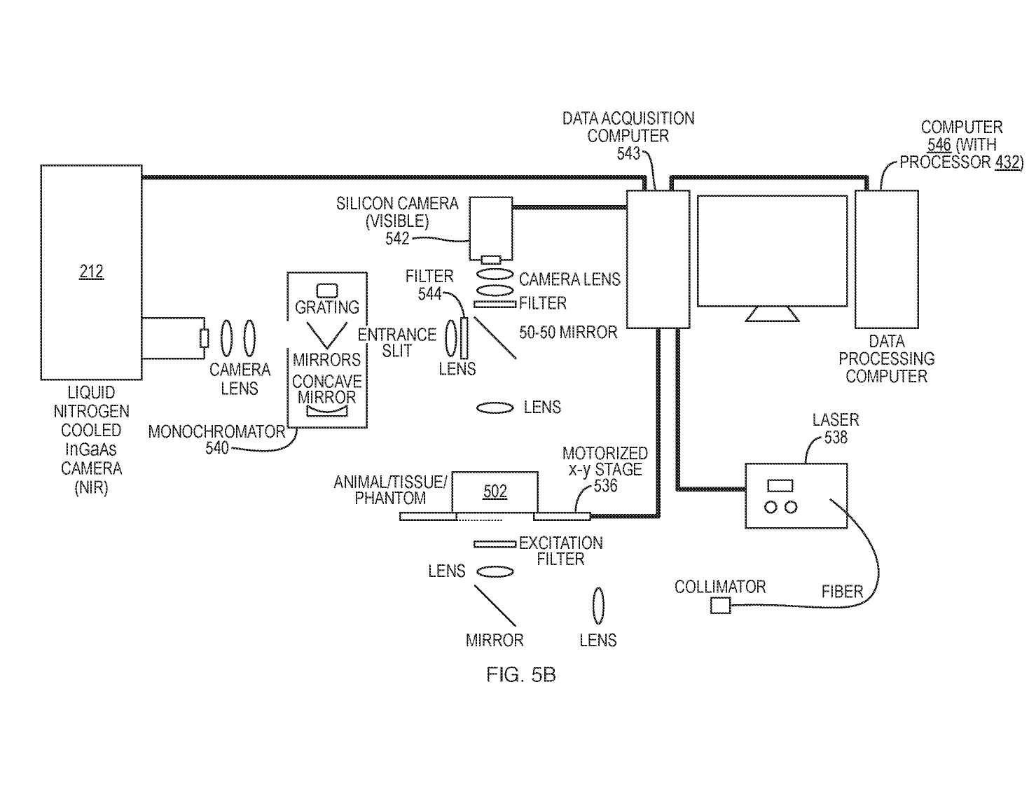

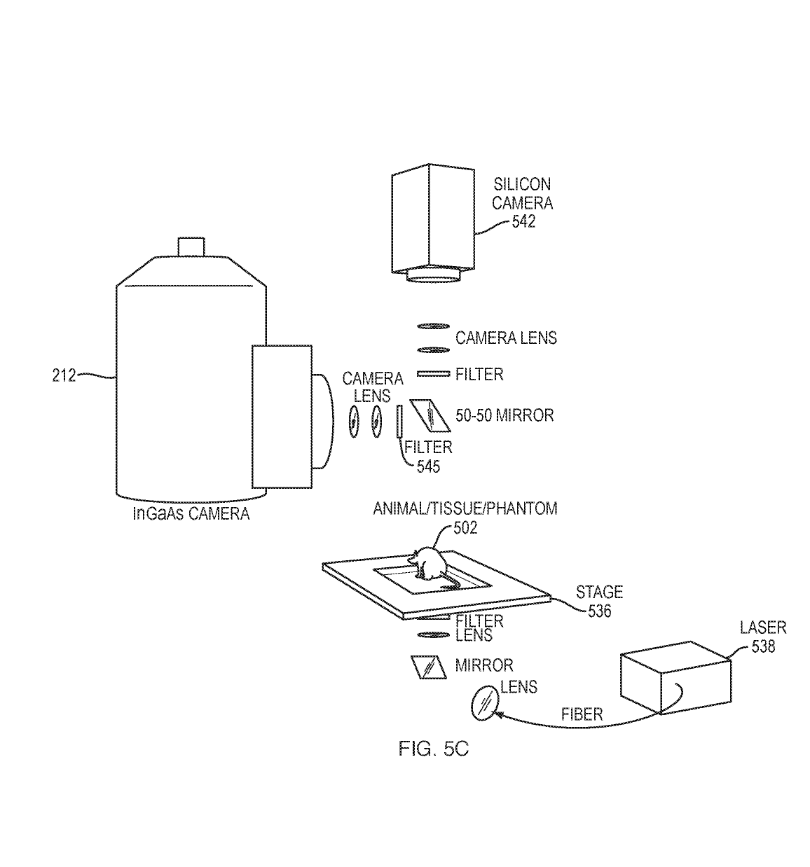

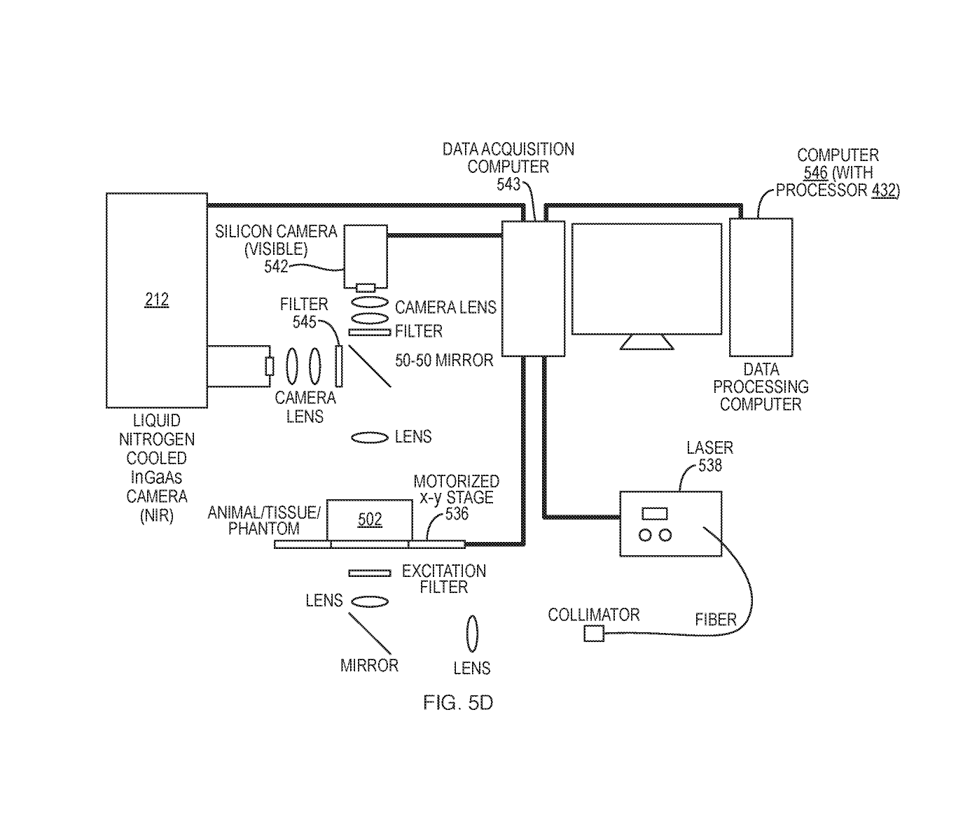

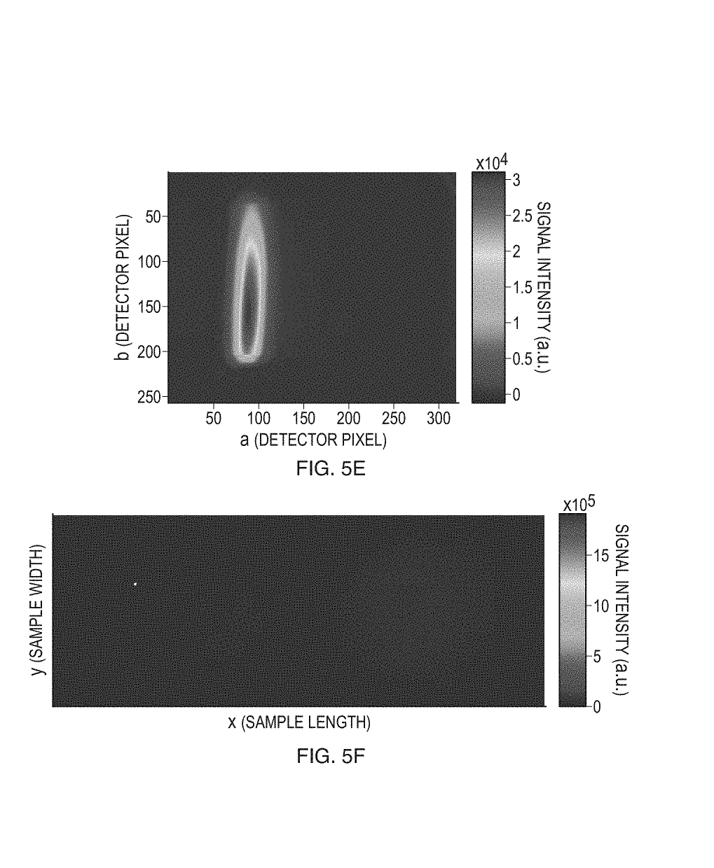

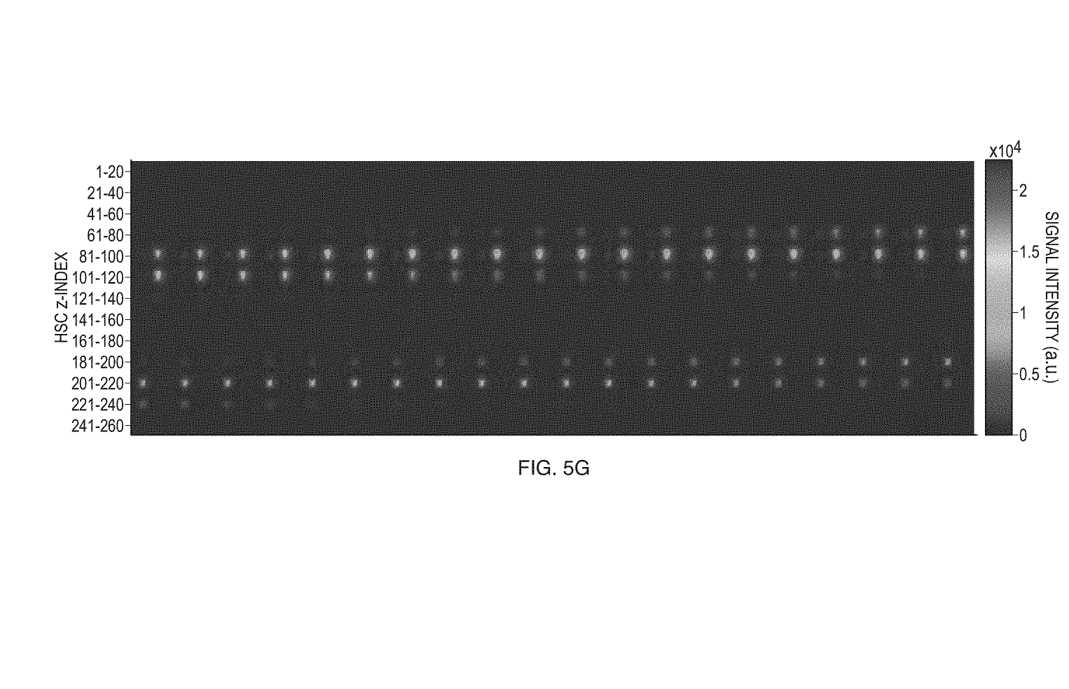

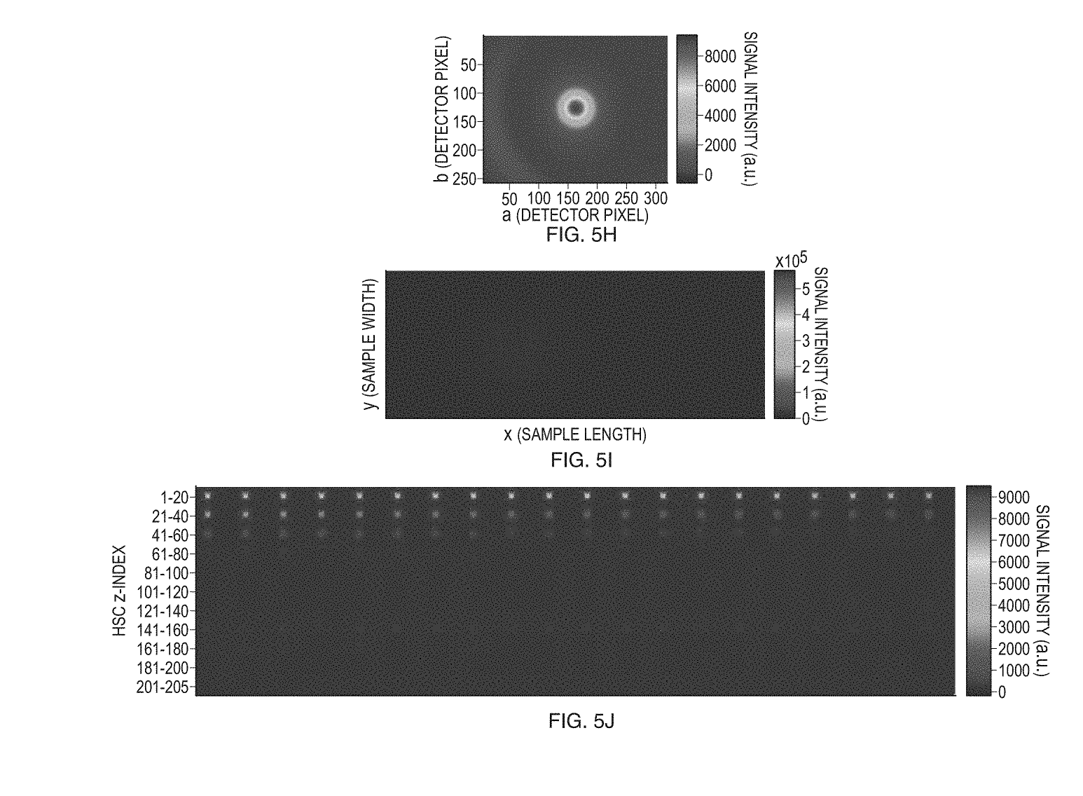

FIG. 2B also illustrates how hyperdiffuse imaging can be performed based on information obtained from the hyperspectral imaging. Namely, at 222a, hyperdiffuse imaging can be performed for each wavelength spectral band component identified at 220c based on the PCA of the hyperspectral image data. An optical bandpass filter can be employed for each spectral band component, for example, as further described in conjunction with FIGS. 5C-5D.

At E, raw hyperdiffuse imaging data are processed to form a hyperdiffuse cube (HDC) at 222b, per the following expression:

I_{α-δ}(x,y,a,b)→I_{α-δ}(x,y,r):HDC_{α-δ}(x,y,r) E:

where

r={square root over (α|−α_{c})²+(b−b_{c})²)}

is the radial distance from (ac, bc), a center position predetermined during alignment of the illuminating laser spot size with the center pixel of the image frame on the detector.

For the case of hyperdiffuse imaging (HDI), HDI can be performed for each of the above-mentioned spectral bands α-δ, using bandpass filters. From the raw data, the directly measured results I_{α-δ}(x, y, a, b) at 100×100 positions (x, y) comprised of 320×256 intensity pixels (a, b) can be transformed to diffuse imaging of 205 diffuse frames, a hyperdiffuse cube HDC_{α-δ}(x, y, r). r is the distance between pixel (a, b) and the center pixel (ac, bc) corresponding to the incident beam location on the XY plane.

At F, a band-wise PCA is performed to identify diffuse scattering properties at 222c, per the following equation:

[Coeff(λ),Score(x,y),Explained,μ(r)]=PCA(HDC_{α-δ}(x,y,r)) F:

with the functional domains for the four parameters defined as with the functional domains for the four parameters defined as Coeff_{α-δ} (λ, rank=1-4); Score_{α-δ}(x, y, rank=1-4); Explained_{α-δ}(rank=1-4); and μ_{α-δ}(r).

The first parameter, Coeff, contains information describing the transformation of principal components from diffuse frames. Coeff of the first component is plotted to identify the most pronounced contributions from diffuse frames. The second parameter, Score, contains the linear combination processed image for the first principal component, indicating the image with highest contrast obtained from linear combination of diffuse frames. The third parameter, Explained, describes the contribution from each principal component to the measured results, HDC(x, y, r). The first component from PCA always dominates the HDC, by definition, because the principal components are designated in descending order. Note that in extreme cases, for example when two sources have the exact same emission intensities, the first and second principal components may be equal. Finally, the fourth parameter μ is the averaged intensity from each diffuse frame.

At G, pixel-wise diffuse property analyses are performed. Similar to pixel-wise analysis of HSC, pixel-wise analysis of HDC results in diffuse intensity and diffuse width (scattering) information for each pixel, denoted as DIᵢ and DWᵢ:

### G:

$${DI}_{i = α - δ} ( x , y ) = {max}_{r} ( {HDC}_{i = α - δ} ( x , y , r ) ) OR$$

$${DI}_{i = α - δ} ( x , y ) = {median }_{r} ( {HDC}_{i = α - δ} ( x , y , r ) ) OR$$

$${DI}_{i = α - δ} ( x , y ) = {PCA Score }_{r} ( {HDC}_{i = α - δ} ( x , y , r ) )$$

$${DW}_{i = α - δ} ( x , y ) = {Peak Width }_{r} ( {HDC}_{i = α - δ} ( x , y , r ) )$$

At H, inter-band pixel-wise analyses are performed on the diffuse intensity images to produce inter-band images at 222f, according to the following:

### H:

$${DI}_{i / j} ( x , y ) = \frac{{DI}_{i = α - δ} ( x , y )}{{DI}_{j = α - δ , j \neq i} ( x , y )}$$

is utilized to characterize and maximize the image contrast based on the knowledge of the origin of contrast of each spectral region. One or more of these inter-band images DI_{i/j} can then be selected to maximize contrast and provide X-Y projection information to produce 2-D fluorescent images at 222g. DW images, which are described further hereinafter, can be used to provide z depth information, and the z depth information can be combined with the 2-D fluorescent images at 222g to produce 3-D fluorescent images at 222h

As also indicated in FIG. 2B, bright field imaging can be performed to supplement the HSI and/or HDI inter-band images to accomplish 2-D or 3-D image registration. Namely, at 224a, a Silicon camera is used to obtain a bright field image of the target, which is combined with the 2-D fluorescent images to 220h or 222g for 2-D image registration. In addition, or alternatively, a 3-D scanner can obtain a bright field image at 224b, which can be overlayed on the 3-D fluorescent images to 220i or 222h to accomplish 3-D image registration at 224d.

FIG. 2C is a schematic illustration of various sources of image contrast. The section 210a illustrates laser line absorption contrast. Laser light 214 incident at the target is absorbed more readily at a target feature 216a than elsewhere in the target, and camera 212 detects the feature 216a as image contrast. The section 210b illustrates inelastic scattering contrast, in which a feature 216a at the target scatters inelastically (e.g., Raman scattering) more readily other features in the target and is detected by the camera 210 as image contrast. Section 210c illustrates tissue autofluorescence. Section 210d illustrates probe emission fluorescence, in which a probe 216b introduced in a vicinity of that target fluoresces when exposed to the incident laser light 214, and the fluorescence is detected by the camera 212 as image contrast.

FIG. 2D shows relative intensity of tissue autofluorescence as a function Raman shift. Biological tissues containing lipids scatter inelastically with strong Raman shifts 3,000, 2,800-3,000, 1,440 and 1,300 cm⁻¹, corresponding respectively to the unsaturated ═C—H bond stretch, the saturated —CH₂ asymmetric and symmetric stretches, —CH₂ bend and —CH₂ twist vibrations. This analysis enables us to determine the optimal wavelength of emission for an exogenous contrast agent, for a given source of laser illumination. For example, for a 980 nm excitation source, it is beneficial if the probe emission is not centered close to ˜1,388 nm or around ˜1,135 nm, for mitigating the background.

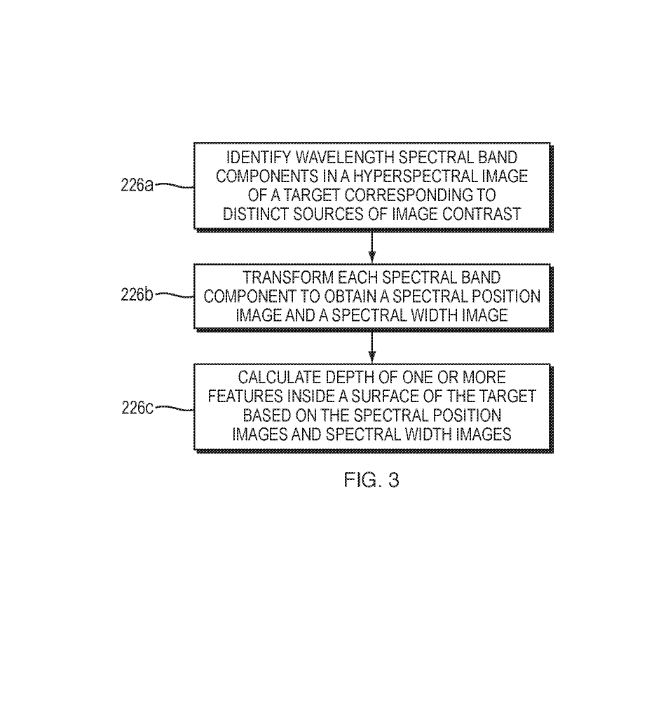

FIG. 3 is a flow diagram illustrating a method of determining depth of features inside a surface of the target. At 226a, a plurality of wavelength spectral band components in a hyperspectral image of a target are identified, where the spectral band components corresponding to mutually distinct sources of image contrast. At 226b, each respective spectral band component is transformed to obtain a spectral position image and a spectral width image corresponding to each respective spectral band component. At 226c, depth of one or more features inside a surface of the target is calculated based on the spectral position images and the spectral width images. In some embodiments, the plurality of wavelength spectral band components can include optical spectral band components.

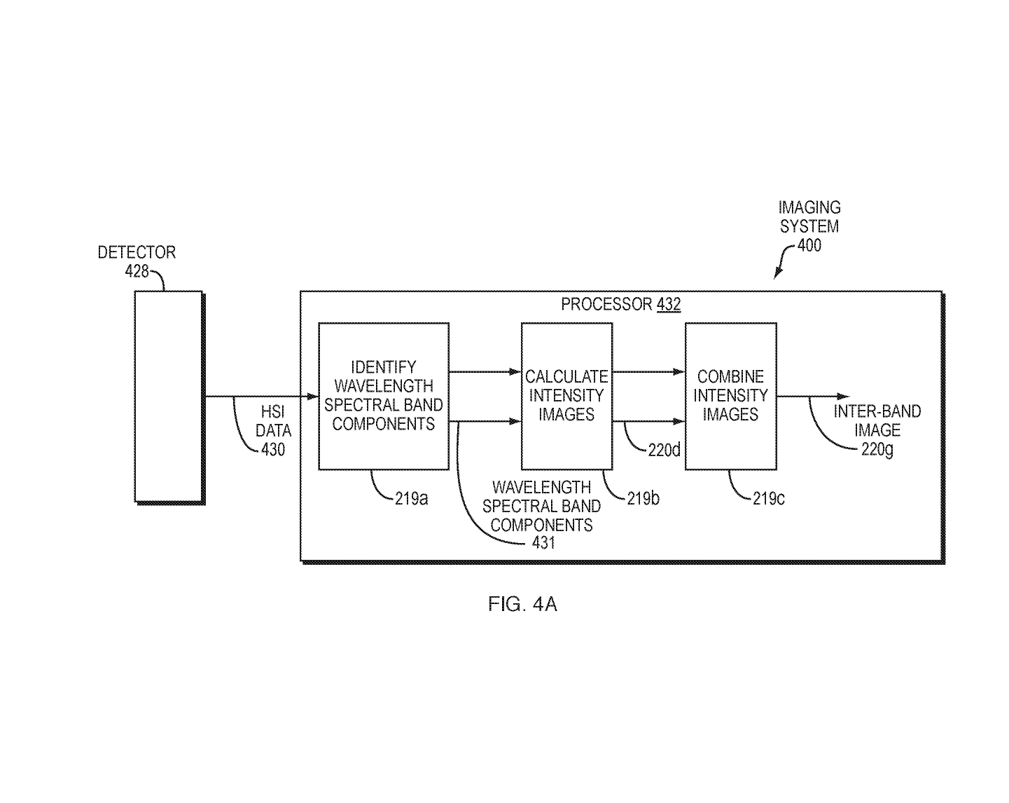

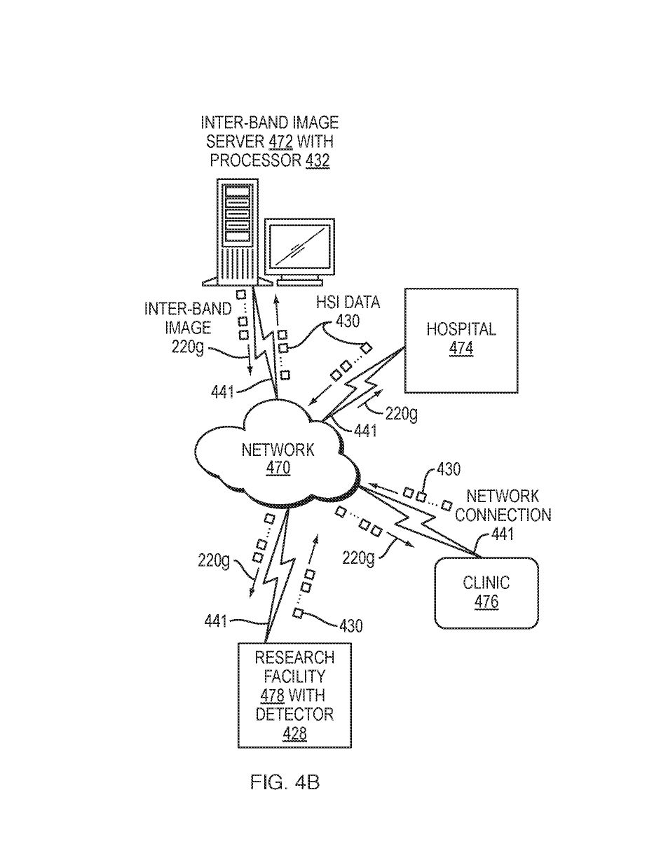

FIG. 4A is a block diagram illustrating an embodiment imaging system 400. The system 400 can be used as part of obtaining images and/or depths according to embodiment methods illustrated in FIGS. 2A, 2B, and 3, for example. A detector 428, which can be an InGaAs detector in some embodiments, is configured to acquire HSI data 430 for a target (not shown). A processor 432 is configured to identify, at 219a, a plurality of wavelength spectral band components 430 in the hyperspectral image data, the spectral band components corresponding to mutually distinct sources of image contrast. The processor 432 is also configured to calculate, at 219b, respective intensity images 220d corresponding to each respective spectral band component 430 and to combine, at 219c, the respective intensity images to form an inter-band image 220g based on the respective, mutually distinct sources of image contrast for each spectral band component.

It should be noted that “identifying” a plurality of wavelength spectral band components, as used herein, can include actually analyzing HSI data to determine the components using principal component analysis, for example. Alternatively, “identifying” can include only receiving information about wavelength spectral band components from internal memory, data storage, or from a source external to the computer, for example.

In FIG. 4A, all necessary processing is performed by a single processor 432. However, in other embodiments, functions performed by the processor 432, including 219a, 219b, and 219c, can be divided among any number of processors. The HSI data 430 can include ray HSI data 220a or HSC data 220b (both illustrated in FIG. 2B), for example. In some embodiments, such as that described hereinafter in conjunction with FIGS. 5A-D, a system further includes capability to actively illuminate a target, such as with laser, to obtain HSI and/or HDI image data. Wavelengths of incident laser light can be in a range between about 750 nm and about 1600 nm. Preferably, the wavelengths of incident light are between about 750 nm, such as 750±50 nm, and about 1100 nm, such as 1100±50 nm. Specific wavelengths of incident light can include, for example, 808±5 nm, 980±5 nm, 1064±5 nm, and 1550±5 nm. Wavelengths of incident light can be chosen such that there is a wavelength separation between incident light, light inelastically scattered from the target medium, tissue autofluorescence due to Raman scattering effects caused by the interaction of the light with the tissue environment, and any probe emission wavelength. Thus, distinguishing the probe signal from excitation or autofluorescence using idealized optical band-pass filters can be facilitated. For example, one suitable configuration would be to use a 980 nm laser excitation source, with an Er-doped UCNP as the exogenous contrast agent, with a peak emission at ˜1,575 nm, which avoids the Raman scattering effects caused by the 980 nm laser at ˜1,350 nm. In this case, it is possible to use a combination of filters: 2×1,500 nm long-pass filters, and 2×1,575 nm±25 nm bandpass filters to distinguish the probe emission, while excluding the excitation or autofluorescence background.

Two major factors determine the maximal penetration depth that an optical imaging system such as system 400 in FIG. 4A can achieve: (1) signal-to-noise ratio (SNR), affected particularly by the level of attenuation of the signal from the probe emission by tissue, and the level of noise generated by the tissue upon excitation by the laser directly or by the probe emission indirectly; and (2) diffuse scattering of the probe emission upon travel through deep turbid biological tissue, affected by the tissue properties (μₐ, μₛ′, g, n) and the wavelength and directionality of all light sources (excitation, emission and autofluorescence). A modularized optical imaging system has been built to study these two factors, as described hereinafter in the description of FIGS. 5A-D.

FIG. 4B is a schematic diagram illustrating a network 470 in which embodiment systems and methods can be employed. In particular, FIG. 4B illustrates an inter-band image server 472 containing the processor 432 of FIG. 4A. The server 472 receives the HSI data 430 via network connections 441 from a hospital facility 474, a clinic facility 441, and a research facility 478 in which embodiment systems are employed. The server 472 performs the functions described in connection with processor 432 in FIG. 4A and returns (transmits) inter-band image data 220g to the respective facilities according to the respective HIS data 430 received via the various connections 441.

The network connections 441 can include, for example, Wi-Fi signals, Ethernet connections, radio or cell phone signals, serial connections, or any other wired or wireless form of communication between devices or between a device and the network connections 441 that support the communications. Network-server-based embodiments such as that illustrated in FIG. 4B can be used with a subscription service. Client facilities such as hospital facility 474, the clinic facility 441, and the research facility 478 can house embodiment systems, such as those described hereinafter in connection with FIGS. 5A-5D, to acquire HSI data. These facilities can upload the data to the server 472 and then receive inter-band images, which can be provided for free or for a subscription payment, for example.

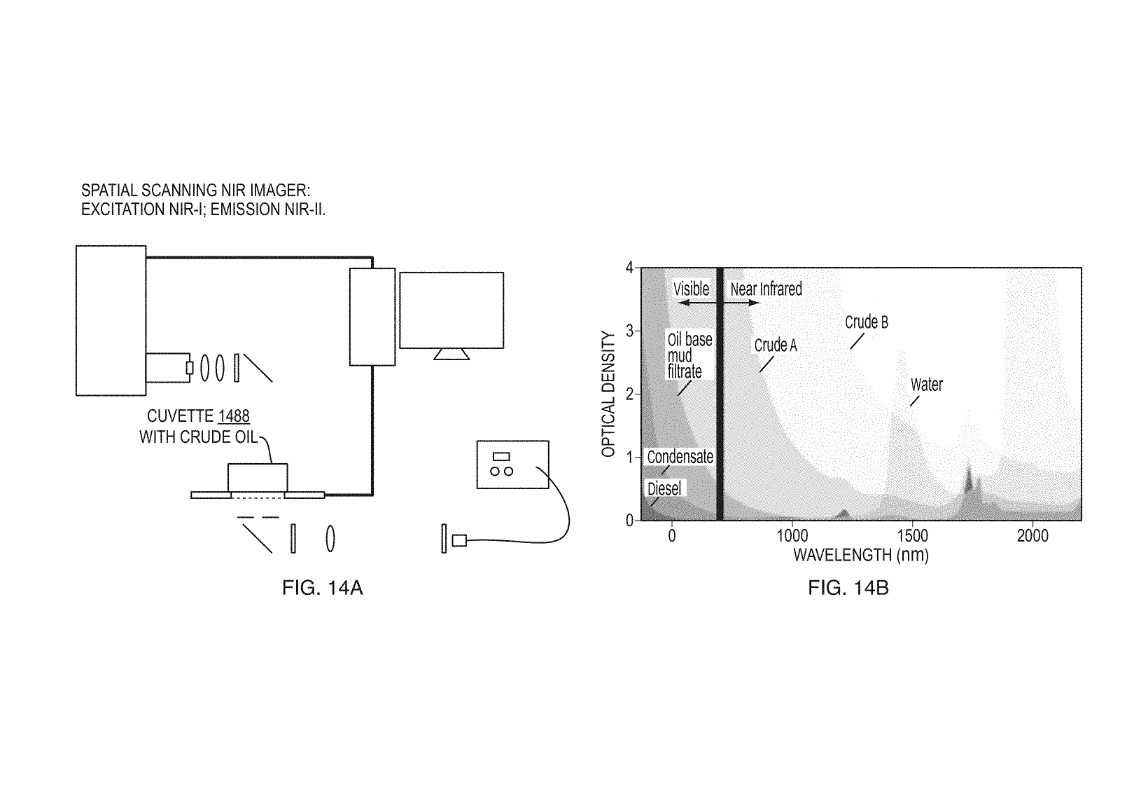

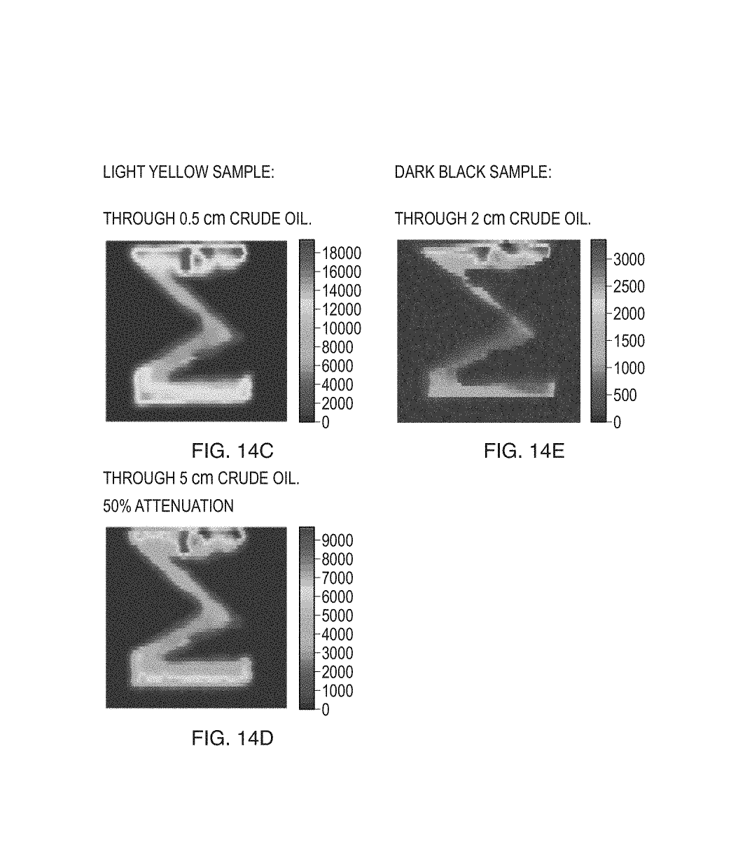

FIGS. 5A-5D illustrate a system for Detection of Optically Luminescent Probes using Hyperspectral and Diffuse Imaging in Near-infrared (NIR) imaging (DOLPHIN). This system is designed for imaging specifically in the NIR region for deep tissue penetration, and this system has been called a “Detection of Optically Luminescent Probes using Hyperspectral and Diffuse Imaging in Near-infrared” (DOLPHIN). The target medium of interest can be the whole body, organ or tissue, tumor microenvironment or cellular level imaging, as well as tissue “phantoms” designed to mimic the optical properties of biological tissues, and cell culture in vitro. Furthermore, systems such as those illustrated in FIGS. 5A-D can also be used for deep imaging of other organic targets, such as fossil fuels, as described hereinafter in conjunction with FIG. 14.

Still referring to FIGS. 5A-D, Light from a laser 538 is coupled through a fiber and collimated by a lens, reflected by a mirror up though a lens and filter through a stage 536 to be incident at a mouse target 502. The stage 536 is motorized to allow for scanning the target 502 with respect to the laser light beam. However, in other embodiments, the laser light can be scanned and the target can be stationary, for example. The target can also be a human, other animal, other organic target, etc. The laser light, as well as any fluorescent light or scattered light, is reflected by a 50-50 mirror toward camera lenses 534 and eventually detected by the InGaAs camera 212, which includes a detector such as the detector 428 in FIG. 4A. A separate silicon camera 542 is configured to acquire a bright-field image of the target 502 for image overlay and 2-D registration purposes.

The imaging platform, as depicted by the black stage in FIGS. 5A and 5C, can be used for imaging whole animals, tissues or organs, cells in vitro or tissue mimic phantoms. The DOLPHIN system has been depicted here in forward imaging mode, in which the incident laser passes through the specimen in a transillumination configuration. The target medium is in the optical path between the laser light source and the detector array in the InGaAs camera. Alternatively, the instrument can also be configured in reflectance or angular imaging modes, in which the laser is incident from the same side as the silicon camera (near-coaxial illumination) or at an angle of 0-180° with the specimen stage, respectively. In reflectance or angular imaging modes, the detector can be positioned to substantially avoid detection of light from the laser illuminating the target medium, as understood in the art of spectroscopic imaging.

In DOLPHIN, a collimated laser beam in NIR-I (such as 808 nm or 980 nm) is pre-aligned with light collection elements in transillumination configuration shown in FIG. 5A. In this configuration, also termed as “forward imaging mode”, the target medium being imaged is located in an optical path between the illuminating light source, and the detector array. Alternatively, the system could also be configured in “reflectance imaging mode” (not shown here), wherein the illuminating laser is located on the same side as the detector array (nearly coaxial), with the image being collected after interaction of the irradiant light with the target medium and other features of interest. Alternatively, the system could also be configured in “angular imaging mode” (not shown), in which the illuminating laser is located in an optical path between the target medium and the detector array, at an angle ranging from 0° to 180° between the incident and transmitted light.

The system illustrated in FIGS. 5A-5D has the ability to operate in two modes, namely, hyperspectral imaging (HSI) and hyperdiffuse imaging (HDI) modes. In the HSI mode (FIGS. 5A-5B), the collection light path is composed of collection lenses, a monochromator spectrograph with NIR diffraction gratings to collect the entire wavelength spectrum from 800-1,700 nm (2.5 nm/pixel), and a liquid nitrogen (LN)-cooled InGaAs camera detector with a 320×256 pixel array, with imaging lenses. In HDI mode (FIGS. 5C-5D), the collection light path is composed of only the camera and imaging lenses, with optical bandpass filters corresponding to the respective spectral band components for which HDI is being performed, each spectral band and corresponding diffuse image being obtained in turn. The excitation source and (spectral) imaging components are fixed, while the tissue sample with probe is placed on an automated XY translational stage with a step resolution of 1 μm in both directions. The 2-D spatial scanning operation on the tissue sample improves spatial resolution and allows us to study and decouple the effect of diffuse scattering from probe fluorescence.

The source of the image contrast can be attributed to either endogenous contrast or exogenous contrast. Sources of endogenous contrast could include the collection of photons from a self-luminous (self-emitting) target medium, such as a bioluminescent organism expressing oxidative enzymes such as luciferase, or fluorescent proteins such as green or red fluorescent protein (GFP, RFP) and certain fluorescent proteins that emit NIR I wavelengths. In the case of target media expressing such endogenous contrast mechanisms, DOLPHIN can be designed to perform HSI/HDI without the need for an external illumination.

Alternatively, in cases where the target medium does not express an endogenous contrast agent, an exogenous contrast agent may be induced externally, such as a fluorescent probe or a molecularly targeted reporter. Examples of these include an organometallic compound, doped metal complexes, up-conversion nanoparticles (UCNPs), down-conversion nanoparticles (DCNPs), single-walled carbon nanotubes (SWCNTs), organic dyes or quantum dots (QDs). In this case, it is necessary to illuminate the fluorescent probe externally, using an NIR-I laser described above, in either forward, reflectance or angular imaging mode. It should be noted that a probe used with embodiment systems and methods described herein can have more than one emission wavelength.

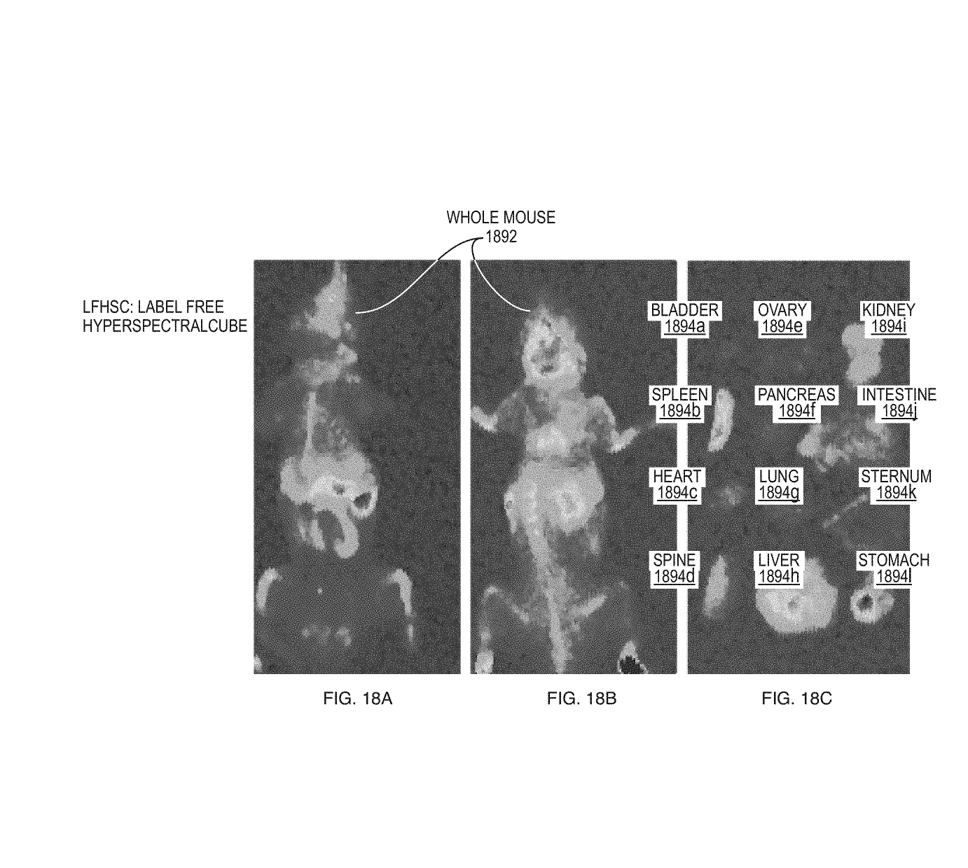

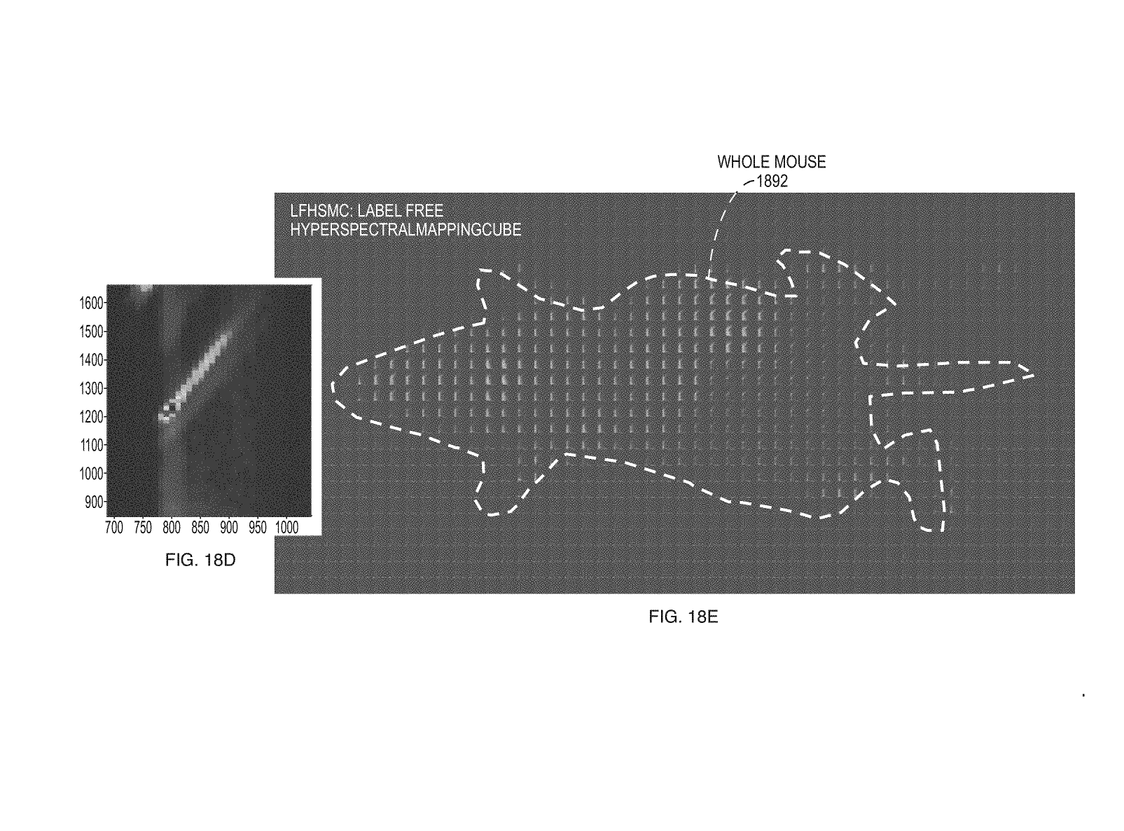

Furthermore, as described hereinafter in connection with FIGS. 18A-18E, imaging using embodiment methods and devices can extend to obtaining hyperspectral image data without exogenous or endogenous labels, by relying on inherent heterogeneities in a target subject being imaged. In such a case of inherent heterogeneities, spectral band components corresponding to mutually distinct sources of image contrast can result from the heterogeneities in the subject represented in the hyperspectral image data. Further, in cases where hyperdiffuse image data such as hyperdiffuse intensity images are available, the spectral band components corresponding to mutually distinct sources of image contrast can result from the heterogeneities in the subject represented in the hyperdiffuse image data

There is also a silicon camera and a 3-D scanner coaxially mounted on the DOLPHIN system, in the optical path between the target medium and the detector. These offer bright field imaging in 2-D and 3-D respectively, and coupled with the 2-D and 3-D fluorescent images from the analyses of the HSI/HDI data, can be overlaid to generate anatomical co-registration. This capability facilitates identification of the 3-dimensional location and spatial distribution of features of interest in the target medium, which can include one or more instances of the determination of lateral (xy) location, z-depth, presence of tumors, vasculature, immune cells, foreign materials such as exogenous contrast agents, or tissue inhomogeneity due to changes in microenvironment in the target medium. In other embodiments, a 3-D scanner is not coaxially mounted on the DOLPHIN system. For example, the 3-D scanner can be a stand-alone entity, and image registration of the 3-D scan data (point cloud) with the HSI/HDI data can be achieved through three non-collinear points defined on the specimen being imaged, with reference to a common pre-defined XYZ coordinate system.

FIGS. 5A-5B show the system operating in HSI mode, so light passes through a monochromator 540 before being detected at the InGaAs camera 212. The presence of the monochromator 540 with a grating element, collecting photons in the wavelength range 900-1,700 nm, distinguishes the HSI mode from the HDI mode, as depicted by the light gray box in and the optical element between the 50-50 beam splitter and the camera lenses in FIG. 5B. FIG. 5B is a schematic illustration of the system shown in FIG. 5A. FIG. 5B additionally shows a data acquisition computer 543 configured to receive image data from cameras 212 and 542 and to control the laser 538 and motorized XY stage 536. A separate computer 546 with processor 432 is configured to further process HSI and HDI image data according to the steps illustrated in FIG. 2B. However, in other embodiments, the further processing can be performed in computer 543 or distributed among other computers or processors not shown in FIG. 5B.

FIGS. 5C-5D illustrate the same system as in FIGS. 5A-5B, except that the system is configured to operate in hyperdiffuse imaging (HDI) mode. Thus, the monochromator 540 is not used in FIGS. 5C-5D. However, a band-pass filter 545 is used so that the camera 212 collects light corresponding to only one principal wavelength component at a time.

FIGS. 5E-5G illustrate multi-level data processing for images captured in HSI mode, namely signal intensity as a function of XY position (FIG. 5E), level I processing of the data corresponding to the xy scanning (FIG. 5F), and level II processing of the image data corresponding to wavelength separation (FIG. 5G). An “MIT”-shaped feature was generated by applying a coating of up-conversion nanoparticles on a quartz glass slide. The three letters were coated in three different UCNPs, corresponding to 3 unique wavelengths of emission. The typical dopant concentrations are NaYF₄:Yb:X=78:20:2 atomic %, with the dopant element X=Er for “M”, Pr for “I” and Ho for “T”, with peak emission at 1,575 nm, 1,375 nm and 1,175 nm respectively.

In HSI mode, FIG. 5E shows a plot of signal intensity as captured by the camera's 320×256 pixel sensor, at a single point of data collection in the rastered grid, with the X-axis corresponding to the wavelength range captured by the monochromator grating (900-1,700 nm) for a frequency-domain resolution of 2.5 nm/pixel. FIG. 5F shows Level 1 processing of the data, which corresponds to the physically rastered grid scanned by the motorized stage assembly, covering the “MIT” feature on the glass slide. The varying signal intensities of the 3 letters are attributed to the variation in the PL quantum yields of the 3 dopants. FIG. 5G shows Level 2 processing of the data, which plots the “MIT” feature after wavelength separation from 900-1,700 nm. There are 320 individual plots with the top left corresponding to 900 nm, and the bottom right corresponding to 1,700 nm. Note that the individual letters light up at their respective peak emission wavelengths.

FIGS. 5H-5J are similar to FIGS. 5E-5G, except that data capture and processing illustrated are for HDI mode in FIGS. 5H-5J, with suitable optical band filters being used to collect the image data in FIG. 5H.

In HDI mode, a similar procedure for data analysis is performed. The key difference here is that, based upon the analysis of the HSI data, a suitable optical filter is selected to perform HDI imaging. For the three “M,” “I,” and “T” features listed above, the combination filters used are (1,400 nm long-pass×2+1,575 nm±25 nm band-pass×2), (1,300 nm long-pass×2+1,375 nm±25 nm band-pass×2) and (1,100 nm long-pass×2+1,175 nm±25 nm band-pass×2) respectively. An example is shown here, for HDI imaging of the “M” letter only.

FIG. 5H shows a plot of signal intensity as captured by the camera's 320×256 pixel sensor, at a single point of data collection in the rastered grid, with both axes corresponding to signal intensity for the narrow band of wavelengths passed by the bandpass filter. FIG. 5I shows Level 1 processing of the data, which corresponds to the physically rastered grid scanned by the motorized stage assembly, covering the “MIT” feature on the glass slide. Only the letter “M” is visible in this case, due to the choice of optical bandpass filter. FIG. 5J shows Level 2 processing of the data, which plots an average of different regions of interest on the camera. There are 128 individual plots, with the top left corresponding to r=2, to the bottom left corresponding to r=256, the radial distance from the center pixel as defined by the central spot of the laser illumination.

### Hyperspectral Cube (HSC)

In order to study the light-probe-tissue interactions taking place in a whole body optical imaging system, HSI mode with spatial scanning capability can be first employed. Transillumination configuration can minimize the incident laser signal—a major contributor to noise or background, and generate balanced emission output at different depths. Thus, transillumination is utilized for deep tissue imaging. Combining an NIR diffraction grating and a 2-D InGaAs detector, hyperspectral information of light-probe-tissue interactions from 900 to 1700 nm are characterized in the form of hyperspectral cube (HSC), with 2-D spatial and 1-D frequency information.

To analyze HSC, principal component analysis (PCA) initially identifies the prominent features in the frequency domain and estimates the relative contributions from each principal component. For hyperspectral images with high signal-to-noise ratio (SNR), PCA creates excellent contrast images by linear combination. The PCA-identified spectral bands are further characterized individually, by peak position and peak width analyses. Small changes in peak position and width reveal the environment surrounding the fluorophores, and enhance image contrast significantly if the probe fluorescence partially overlaps with tissue autofluorescence. In addition, recognizing the photo-physical origin of each band, band division processing is demonstrated to be more efficient for contrast enhancing than linear combination from PCA.

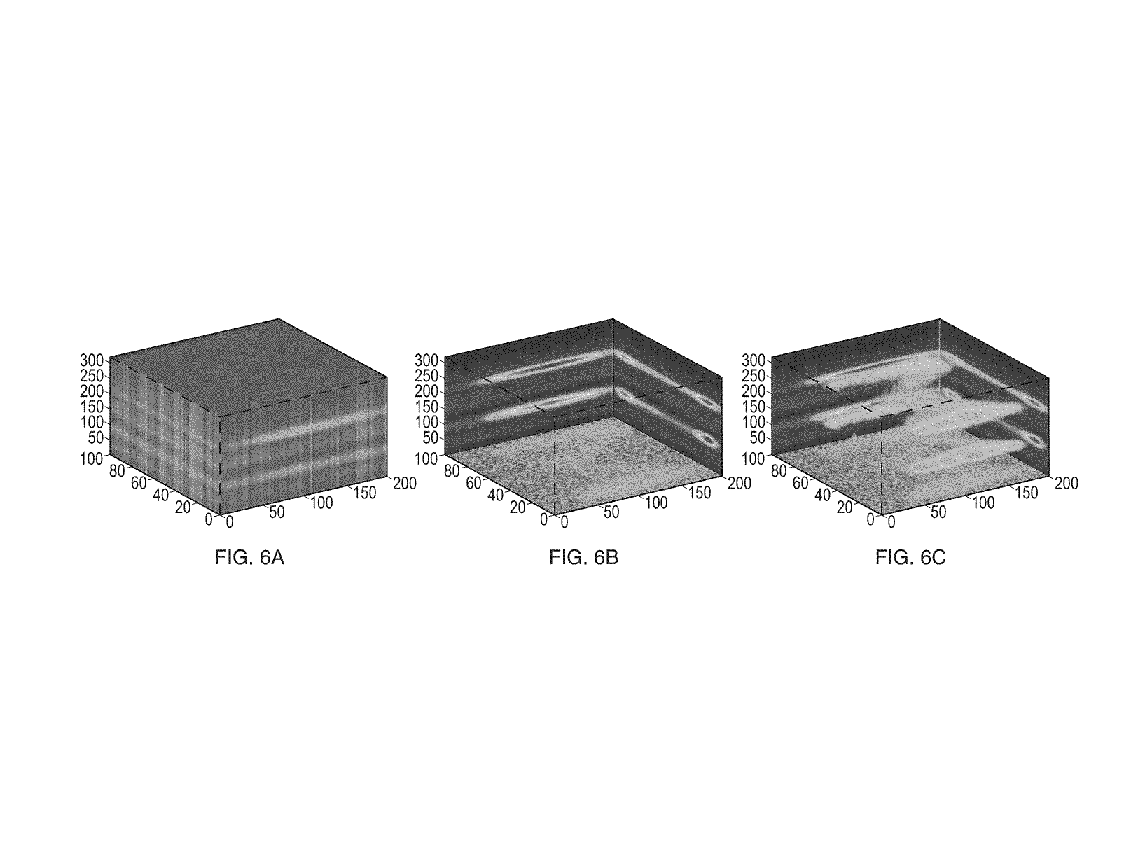

FIGS. 6A-6C are hyperspectral cube (HSC) representations of image data for an “M”-shaped feature. The directly measured results (raw data) I(x, y, a, b) at 100×100 positions (x, y) comprised of 320×256 intensity pixels (a, b) were transformed to HSC of 320 spectral bands, HSC(x, y, λ), where A is the wavelength. I(x, y, a, b)→I(x, y, λ, b)→I(x, y, λ): HSC(x, y, λ). FIG. 6A shows raw data for a z(λ)=I(x, y), for a 100×100 size grid on the XY plane. FIG. 6B is the projection on the XY plane showing the “M”-shaped feature, and the projections on the XZ and Y Z planes showing the peak wavelengths of interest. FIG. 6C shows an overlay stack of the emission spectrum of the “M”-shaped feature at the two peak wavelengths in FIG. 6B.

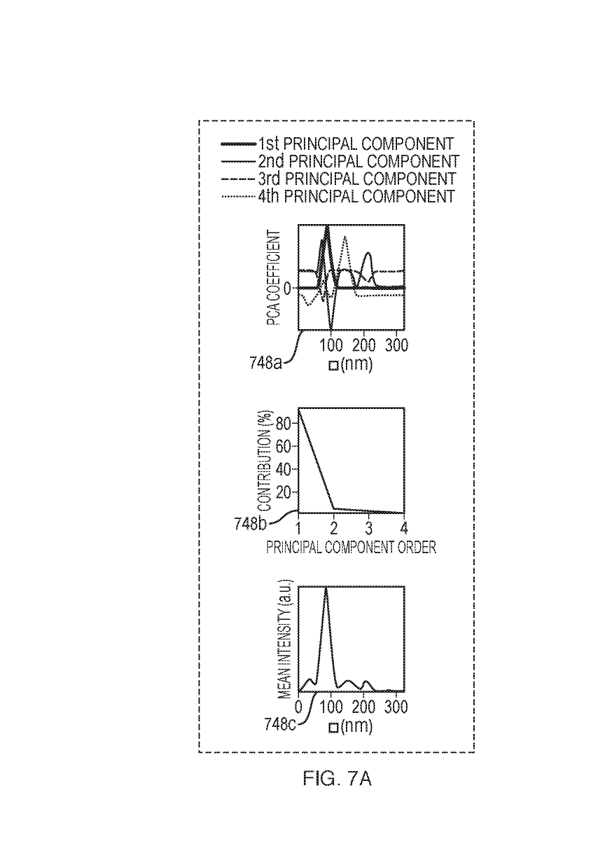

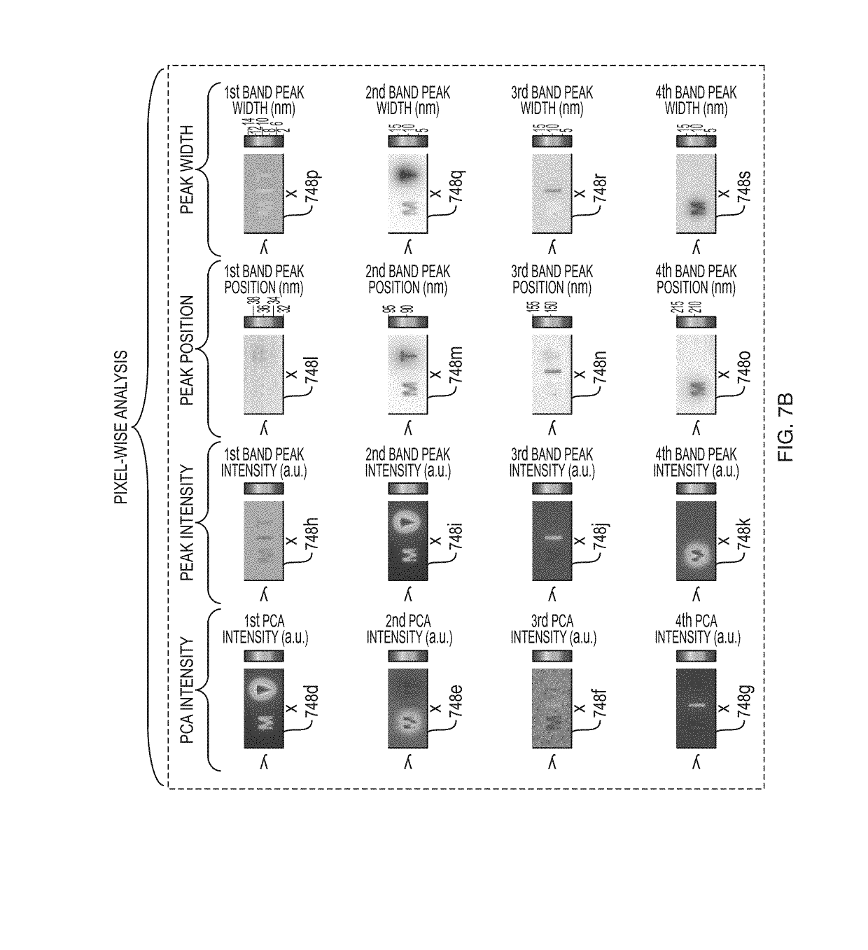

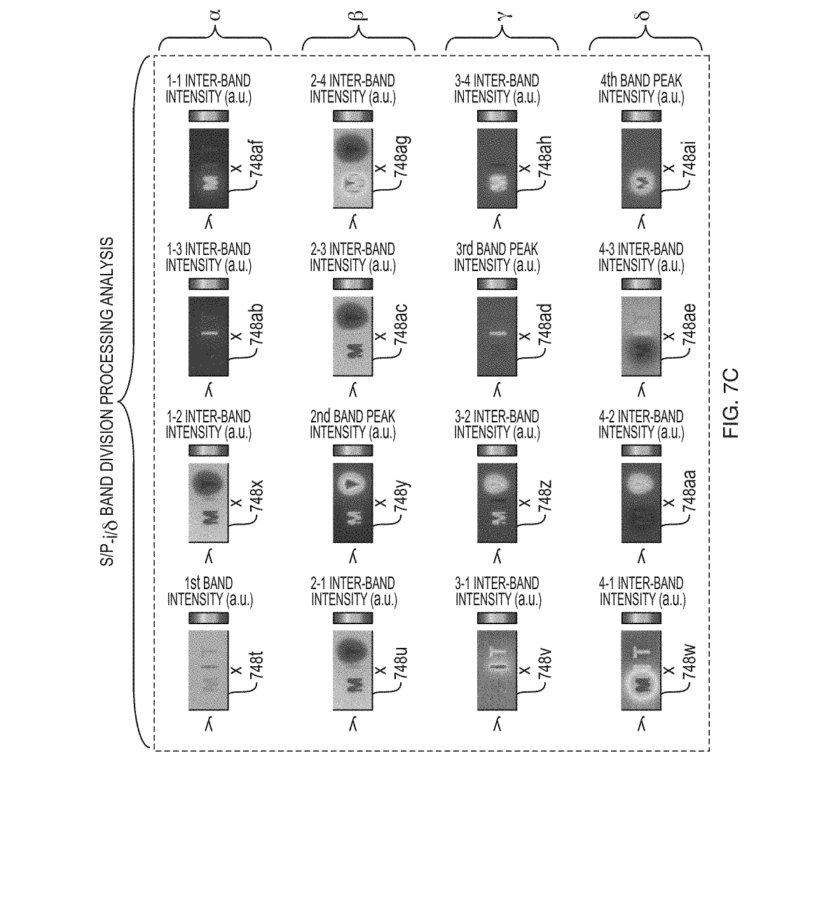

FIGS. 7A-7C illustrate principal component analysis (PCA) applied to hyperspectral cube data (HSC(x, y, λ)) obtained for an “MIT”-shaped feature located at a depth of 20 mm below breast tissue phantom. FIG. 7A shows principal component values as a function of wavelength (748a), contribution of principal components as a percentage of the first component (748b), and band average plotted as the mean intensity (a.u.) as a function of wavelength (748c).

In FIGS. 7B-7C, plots 748d-748ai depict various level 3 data analyses performed on the PCA data. The four rows of plots correspond to the four principal components, identified from PCA: α-band: laser line (absorption contrast), β-band: probe emission I (1100 nm), γ-band: tissue autofluorescence and/or Raman scattering, and the δ-band: probe emission II (1550 nm). FIG. 7B illustrates pixel-wise analysis of the HSC data. Plots 748d-748s represent intra-band pixel-wise analyses performed on the HSC data. The first column, 748d-748g, shows the Score parameter obtained from Score(x, y)=PCA(HSC(x, y, λ)). The second column, 748h-748k, plots the spectral intensity images, calculated as SI(x, y)=PCA Score(HSC(x, y, λ)). Plots 748l-748o show the spectral position images, calculated as SP(x, y)=Peak Position(HSC(x, y, λ)). Plots 748p-748s show the spectral width images, calculated as SW(x, y)=Peak Width(HSC(x, y, λ)).

FIG. 7C illustrates band division processing analysis. Plots 647t-647ai represent inter-band pixel-wise analyses performed on the HSC data; SI_{i/j}(x, y) for i,j=α, β, γ, δ. When i=j, the diagonal elements 748t, 748y, 748ad, and 748ai correspond to the four principal components, which are exactly the same as 748h, 748i, 748j, and 748k, respectively, in the Peak Intensity column of FIG. 7B. The non-diagonal elements provide new information, and this inter-band analysis is used to maximize the image contrast based on the knowledge of origin of contrast of each spectral region. For example, the non-diagonal element 748w, which represents SI_{δ/γ}, offers a much sharper resolution, and consequently a better visualization, of the features of interest, in this case the “MIT” feature, compared to the blurring observed in some of the diagonal principal components.

### Band-Wise Analysis:

For multidimensional data HSC(x, y, λ), performing PCA achieves and presents the most valuable information in lower dimensional space. The following four parameters are obtained:

[Coeff,Score,Explained,μ]=PCA(HSC(x,y,λ))

with the functional domains for the four parameters defined as Coeff(Δ, rank=1-4); Score(x, y, rank=1-4); Explained(rank=1-4); and μ(λ).

The first parameter, Coeff, contains information describing the transformation of principal components from spectral bands. Coeff of the first four principal components (ordered by the relative contribution from each component to the HSC) are plotted (FIG. 7A, graph 748a) to help identify the most pronounced spectral bands. Four bands have been identified based on PCA and the light-probe-tissue interaction, α-band: laser line (absorption contrast), β-band: probe emission I (1100 nm), γ-band: tissue autofluorescence and/or Raman scattering, and the δ-band: probe emission II (1550 nm). The second parameter, Score (FIG. 7A), contains the linear combination processed image from each principal component listed in order of contribution; most information is contained in the first three components and the rest are dominated by noise. The third parameter, Explained, describes the contribution from each principal component to the measured results, HSC(x, y, λ). Depending on the complexity of the tissue sample/probe combination, four principal components can contribute to the original HSC to some extent. In some cases, there can be many more principle components, and this is controlled by the number of individual sources of light in the entire HSI, whether the illuminating laser, inelastic scattering effects such as Raman scattering from the tissue, or emission from exogenous contrast agents. However, this is a deterministic quantity, since it is controlled entirely by the user, and hence, known. For example, if 3 kinds of UCNPs were injected into the same sample tissue, such as the Er-, Pr- and Ho-doped particles, six principal components (one from the laser line, one from the Raman scattering, 2 emission lines from Er-doped, and one each from the Pr- and Ho-doped particles. Finally, the fourth parameter μ is the averaged intensity from each spectral frame, which also serves as an indicator for important bands (more evident for data with high SNR).

### Pixel-Wise Analysis:

Based on four spectral bands obtained from PCA, each HSC is divided into 4 groups:

HSC_{i=α-δ}(x,y,λ(i))

where i denotes the i^{th} spectral band (α-δ). Intra-band analysis is performed on HSCᵢ to achieve pixel information for each band of Spectral Intensity, Position, and Width: denoted as SIᵢ, SPᵢ, and SWᵢ, shown in FIG. 7B (A_{α1} to A_{δ4}) (748d to 748s), where

$${SI}_{i = α - δ} ( x , y ) = {max}_{λ} ( {HSC}_{i = α - δ} ( x , y , λ ( i ) ) ) OR$$

$${SI}_{i = α - δ} ( x , y ) = {median }_{λ} ( {HSC}_{i = α - δ} ( x , y , λ ( i ) ) ) OR$$

$${SI}_{i = α - δ} ( x , y ) = {PCA Score }_{λ} ( {HSC}_{i = α - δ} ( x , y , λ ( i ) ) )$$

$${SP}_{i = α - δ} ( x , y ) = {Peak Position }_{λ} ( {HSC}_{i = α - δ} ( x , y , λ ( i ) ) )$$

$${SW}_{i = α - δ} ( x , y ) = {Peak Width }_{λ} ( {HSC}_{i = α - δ} ( x , y , λ ( i ) ) )$$

As illustrated in the equations above, spectral intensity images can be calculated based on a wavelength of maximum or median intensity, or based on a PCA Score wavelength, as identified in the respective spectral band. Each of these characteristics help identify different features of one spectral region or peak. Further, the ratio

$${SI}_{i / j} ( x , y ) = \frac{{SI}_{i = α - δ} ( x , y )}{{SI}_{j = α - δ , j \neq i} ( x , y )}$$

is utilized to characterize and maximize the image contrast based on the knowledge of the origin of contrast of each spectral region (FIG. 7A, A_{α5} to A_{δ8}) (748t to 748ai).

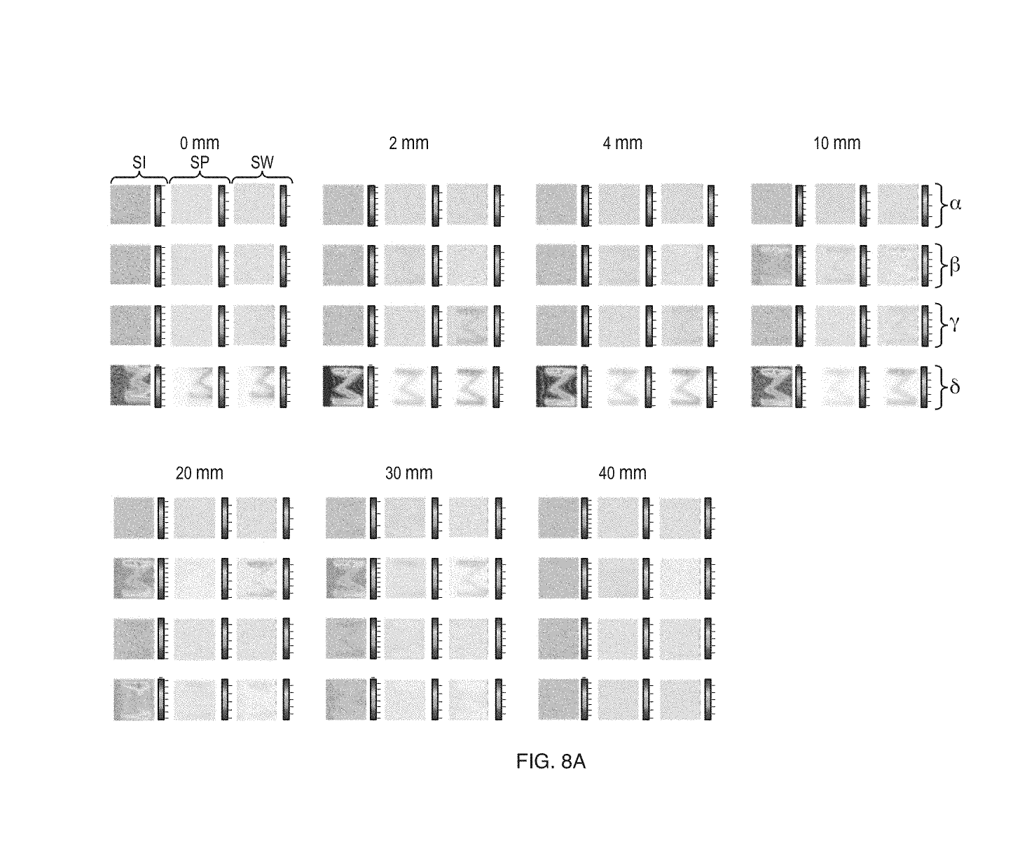

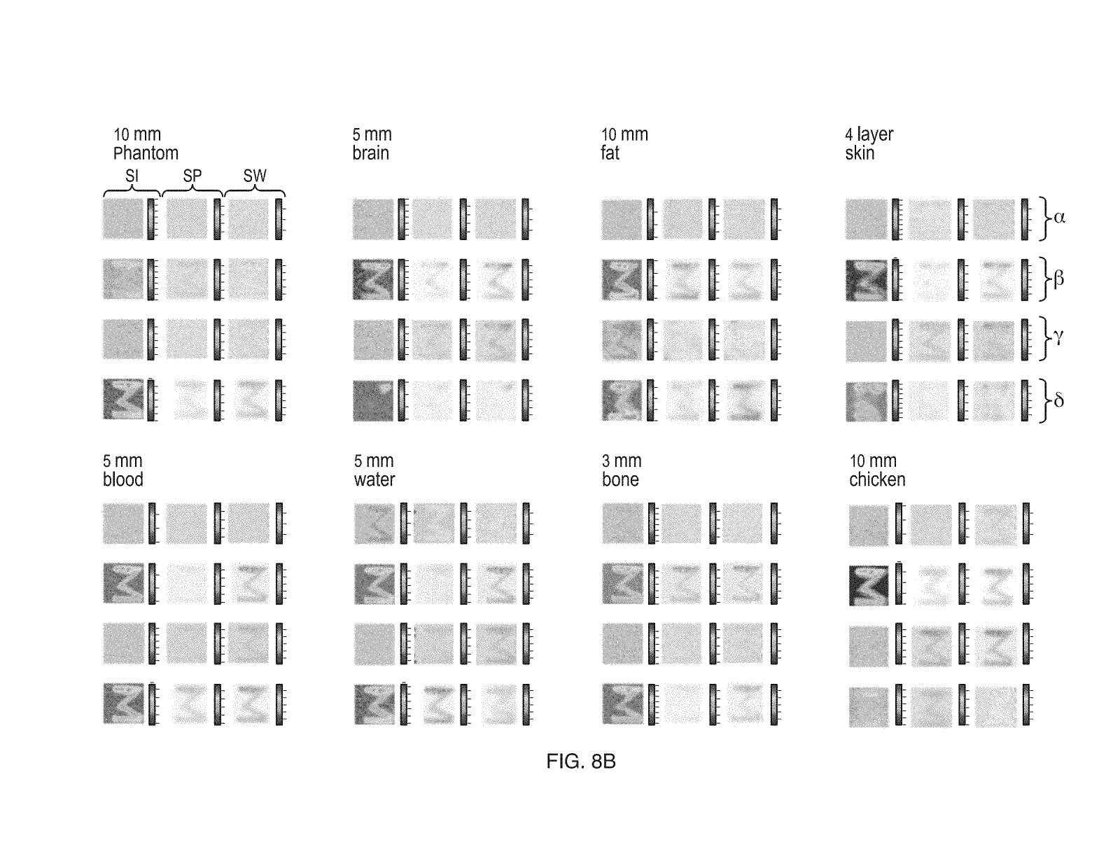

FIG. 8A shows HSI data sets as a function of varying depths in breast tissue phantom from 0 to 40 mm.

FIG. 8B shows HSI data sets as a function of varying tissue properties, studied in phantom, brain, fat, skin, blood, water, bone and chicken tissue.

In both of the data sets of FIGS. 8A and 8B, a 4×3 array is shown for each respective depth or tissue. The four rows correspond to the 4 principal components (α-δ bands), while the three columns represent intra-band pixel-wise analysis performed using SI, SP and SW respectively.

For breast tissue-mimic phantom, hyperspectral imaging of tissue penetration up to 50 mm depths (FIG. 8A) was performed. Positive contrasts of “M” shaped feature show up for emission I (β) and emission II (δ) spectral bands, while negative contrasts show up for laser absorption (α) and tissue autofluorescence (γ) spectral bands. Since the particles emit more efficiently in the δ-band than the β-band and most tissues absorb more in the δ-band (due to water absorption), the δ-band has stronger signals up to 20 mm, while the β-band has stronger signals for more than 30 mm penetration. This phenomenon is understandable, however unintuitive to some extent. This is because most prior studies have employed the δ-band throughout investigation. In contrast, according to the approach used for FIGS. 8A-B, only through HSI in the short-wavelength infrared (SWIR) range can the best imaging condition be determined. Additionally, peak positions and peak widths in both β- and δ-bands show systematic changes, relating to various absorbing features of tissue. Increasing penetration depth results in larger spectral change in peak position and width. On the other hand, these absorbing features arise from small molecules (e.g. water and fat), and different tissue types contain different composition of small molecules, resulting in different inter-band peak intensity ratio change and intra-band peak position and width changes. The HSI of different tissue types suggest that HSI in SWIR is an efficient technique to identifying surrounding environment of probes.

### Hyperdiffuse Cube (HDC)

Another important aspect limiting penetration depth in whole body optical imaging system is the transport scattering effect, normally characterized by diffuse optical tomography or topography. The spatial scanning method allows us to examine the topographical diffuse scattering property pixel-by-pixel. At each pixel, the measured intensity is characterized by the distance from the incident light source (similar to a spatial domain power spectrum). Again, this multi-dimensional data (coined hyperdiffuse cube, HDC) is analyzed by PCA, and the contrast images enhanced by linear combination and contributions from each original component are plotted. Pixel-wise scattering-profile analysis is applied to generate the diffuse imaging results. In the diffuse images, the diffuse scattering property is both penetration thickness and tissue type dependent.

### HDC Result Analysis:

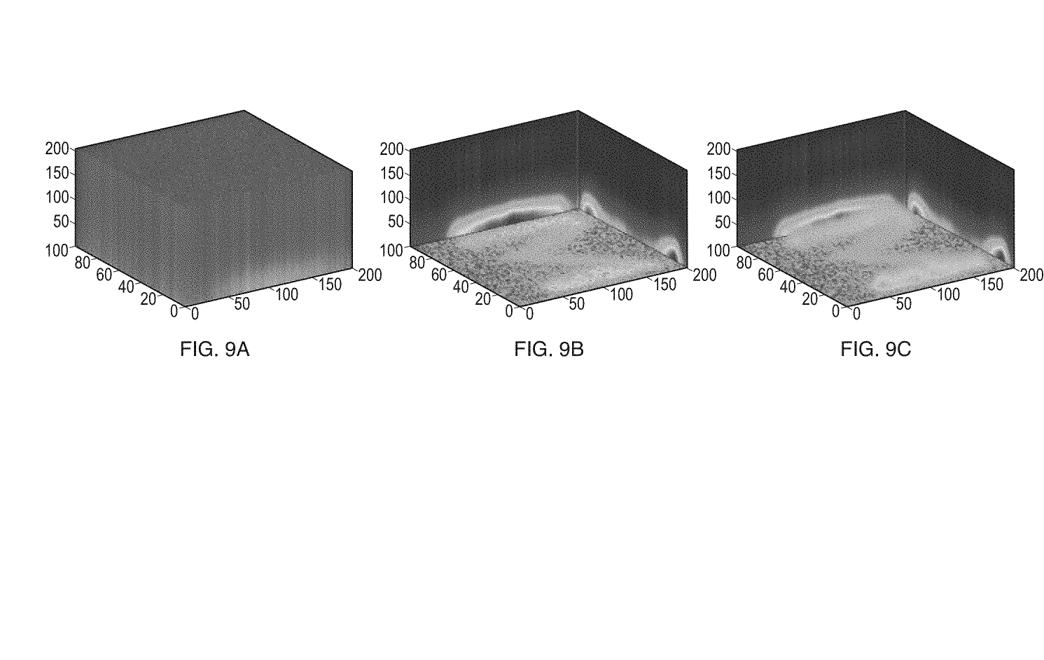

FIGS. 9A-9C show imaging data in the form of a hyperdiffuse cube (HDC). FIG. 9A shows the raw data z(r)=I(x, y), for a 100×100 size grid on the XY plane. FIG. 9B shows a projection on the XY plane of the “M”-shaped feature being imaged. FIG. 9C shows the same projection as FIG. 9B, with a transparency mask added with an opacity function proportional to the intensity of the colormap at each pixel. For each of the above-mentioned spectral bands α-δ, HDI is performed using bandpass filters. From the raw data, the directly measured results I_{α-δ}(x, y, a, b) at 100×100 positions (x, y) comprised of 320×256 intensity pixels (a, b) were transformed to diffuse imaging of 205 diffuse frames, HDC_{α-δ} (x, y, r). The parameter r is the distance between pixel (a, b) and the center pixel (a_{c}, b_{c}) corresponding to the incident beam location on the XY plane.

I_{α-δ}(x,y,a,b)→I_{α-δ}(x,y,r):HDC_{α-δ}(x,y,r)

where r=\sqrt{(a−a_{c})²+(b−b_{c})²} is the radial distance from (aₐ, b_{c}), the center position predetermined during alignment.

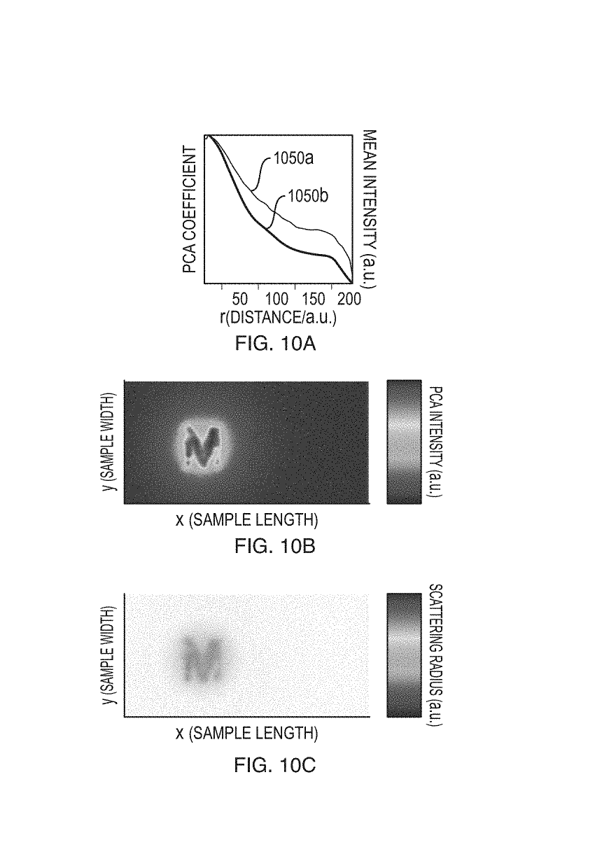

FIGS. 10A-10C illustrate principal component analysis (PCA) applied to HDC (HDC(x, y, r)) data obtained for a “MIT”-shaped figure located at a depth of 20 mm below breast tissue Phantom. FIG. 10A is a graph plotting the PCA coefficient (blue curve) 1050b and the mean intensity (arbitrary units, red curve) 1050a as a function of the radial distance r from the center position (ac, bc) predetermined during alignment of the laser spot. FIG. 10B plots the diffuse intensity images, calculated as DI(x, y)=PCA Score(HDC(x, y, r)), shown here for an optical filter selected for the peak emission of the “M” letter in the “MIT” feature, with the colormap corresponding to the PCA intensity. FIG. 10C plots the diffuse width images, calculated as DI(x, y)=Peak Width(HDC(x, y, r)), with the colormap corresponding to the scattering radius, in arbitrary units.

### Band-Wise Analysis:

For multidimensional data HDC(x, y, r), PCA is performed to achieve and present the most useful information in lower dimensional space.

[Coeff,Score,Explained,μ]=PCA(HDC_{α-δ}(x,y,r))

with the functional domains for the four parameters defined as Coeff_{α-δ} (Δ, rank=1-4); Score_{α-δ} (x, y, rank=1-4); Explained_{α-δ} (rank=1-4); and μ_{α-δ} (r).

The first parameter, Coeff, contains information describing the transformation of principal components from diffuse frames. Coeff of the first component is plotted to identify the most pronounced contributions from diffuse frames. The second parameter, Score, contains the linear combination processed image for the first principal component, indicating the image with highest contrast obtained from linear combination of diffuse frames. The third parameter, Explained, describes the contribution from each principal component to the measured results, HDC(x, y, r) (not shown here, since the first rank is always dominating). The first component from PCA always dominates the HDC. Finally, the fourth parameter μ is the averaged intensity from each diffuse frame.

### Pixel-Wise Analysis:

Similar to pixel-wise analysis of HSC, pixel-wise analysis of HDC results in Diffuse Intensity and Diffuse Width (Scattering) information for each pixel, denoted as DIᵢ and DWᵢ:

$${DI}_{i = α - δ} ( x , y ) = {max}_{r} ( {HDC}_{i = α - δ} ( x , y , r ) ) OR$$

$${DI}_{i = α - δ} ( x , y ) = {median }_{r} ( {HDC}_{i = α - δ} ( x , y , r ) ) OR$$

$${DI}_{i = α - δ} ( x , y ) = {PCA Score }_{r} ( {HDC}_{i = α - δ} ( x , y , r ) )$$

$${DW}_{i = α - δ} ( x , y ) = {Peak Width }_{r} ( {HDC}_{i = α - δ} ( x , y , r ) )$$

Depending on the SNR, different methods (max or median) can achieve similar or even higher contrast DI image compared to PCA analysis (FIGS. 10A-B). Thus, image contrast can be enhanced for a given diffuse intensity image based on a maximum radial distance max r calculated from a plurality of radial distances r, where r is a distance between a given pixel of the respective hyperdiffuse image data and a center pixel corresponding to an incident beam location identified in the respective hyperdiffuse image data. Similarly, contrast may be enhanced based on median r or PCA Score r. A DW image describes the diffuse/transport scattering property (mainly relating to source position, the shape and dielectric environment properties of the surrounding tissue) of each pixel in 2-dimensional projection.

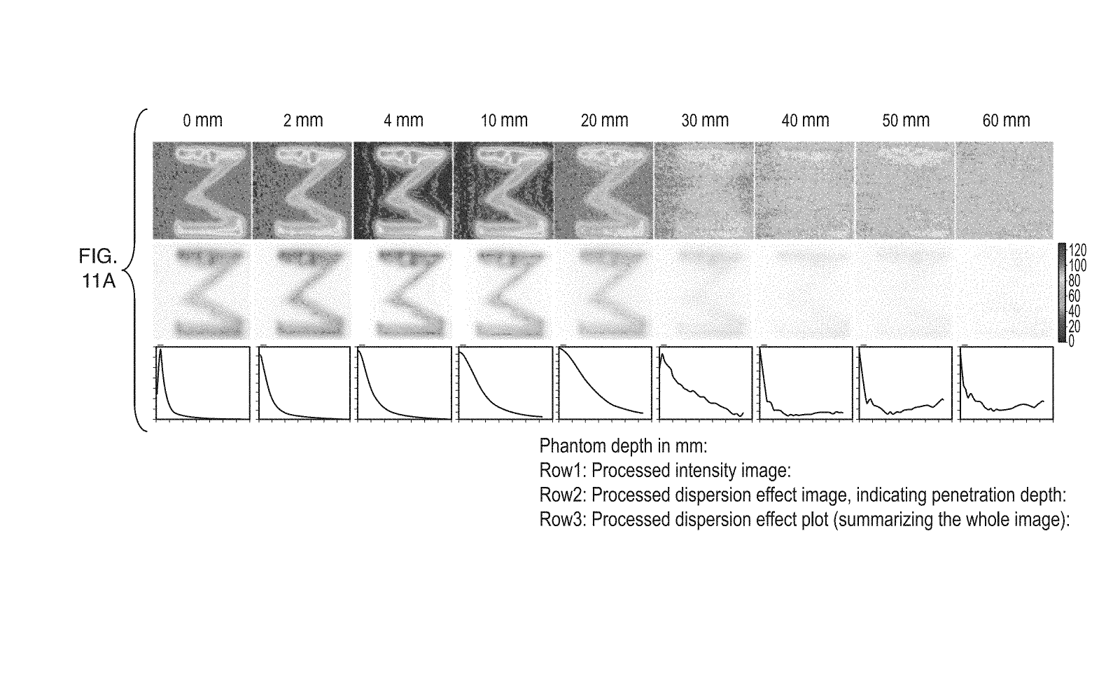

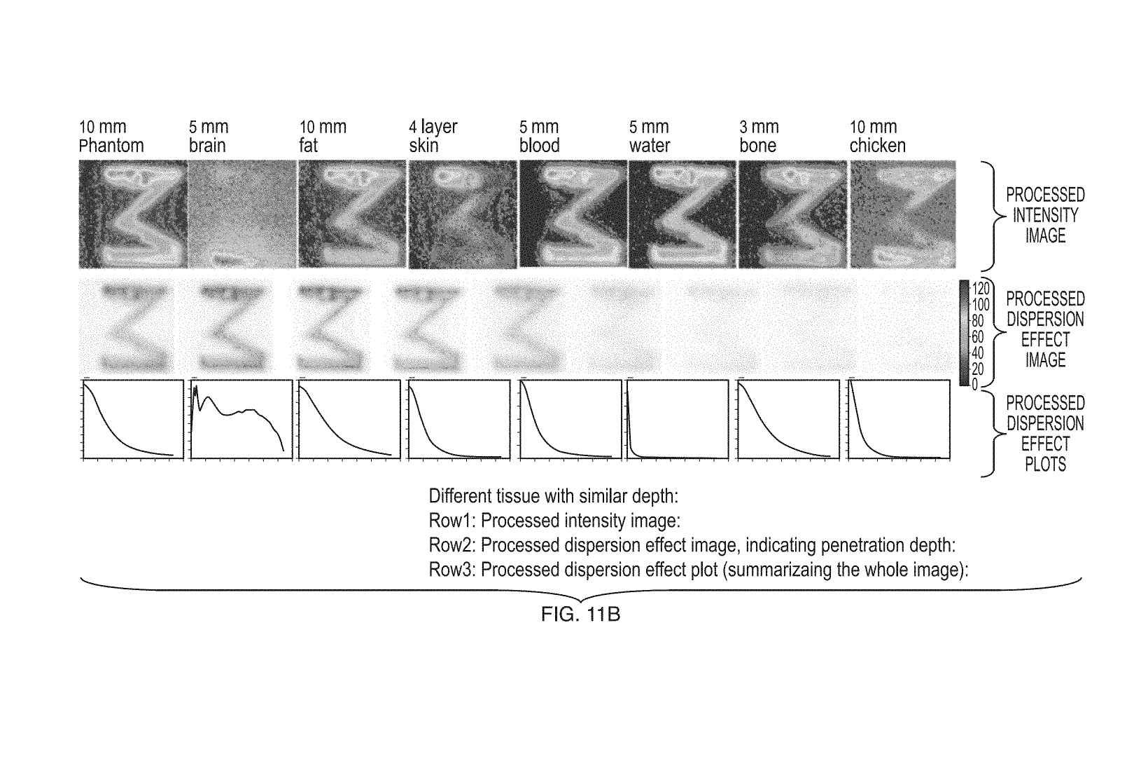

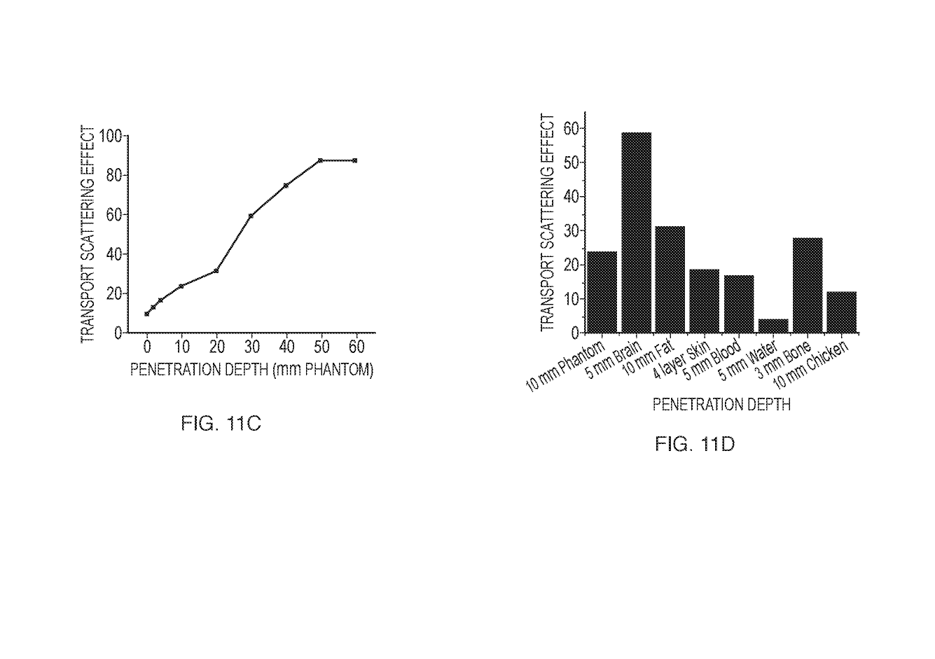

FIG. 11A shows HDI data sets as a function of varying depths in tissue phantom, from 0 to 60 mm and varying tissue properties, respectively. The top row shows processed intensity images (pixel-wise DI analysis) corresponding to the different labeled depths. The middle row illustrates processed dispersion effect images (pixel-wise DW analysis) corresponding to the different respective labeled depths, indicating penetration depth. The bottom row shows processed dispersion effect plots, summarizing the whole image, corresponding to the different respective depths.

FIG. 11B shows HDI data sets as a function of varying tissue properties, studied in phantom, brain, fat, skin, blood, water, bone and chicken tissue, of nearly uniform depth 5-10 mm. For each set of data of FIGS. 11A and 11B, a 3×1 array is shown. The 3 rows correspond to the pixel-wise analysis performed using DI (top row), DW (middle row), and processed dispersion effect plots, respectively.

FIG. 11C is a graph illustrating variations of the transport scattering effect as a function of depth. This graph shows increased transport scattering in deeper tissue.

FIG. 11D is a bar chart illustrating variation of the transport scattering effect as a function of tissue environment, showing strong scattering in brain tissue compared with other types of tissues.

As illustrated by FIGS. 11A-11D, using embodiment methods and apparatus, depth in a range from 0 cm to about 2 cm inside a surface of the target can be determined. Further, depths in a range from about 2 cm to about 3.2 cm or in a range of about 3.2 cm to about 5 cm can be determined. Still further, depths in a range of about 5 cm to about 9 cm, such as depths of 50 mm or 60 mm in FIG. 11A, for example, can be determined.

Hyperdiffuse imaging (HDI) of various penetration depths of breast tissue-mimic phantom (FIG. 7A) show the effect from diffuse scattering of probe emission. As demonstrated by HDI and Monte-Carlo photon migration simulation, it can be observed that probe emission travelling through deep tissues gives rise to a flattened photon fluence on the top tissue surface (photon exiting plane). This results in a broadened emission contour and images without well-defined features, particularly in non-scanning mode. By spatial scanning and processing, the original I_{α-δ} (x, y, a, b) data to HDC_{α-δ} (x, y, r), the diffuse scattering of the probe emission is not only separated and excluded from the resulting contrast image (either by PCA or empirical arithmetic operation), but also the effect of diffuse scattering is identified for each pixel and plotted in a manner similar to diffuse topography (FIGS. 11C-11D). For imaging penetration depth up to 70 mm of breast tissue phantom, it is observed that decreasing detected signal as well as increasing diffuse scattering. While investigating diffuse scattering of different tissue types through HDI, it has been found that most of the tissue types exhibit similar diffuse scattering properties within similar penetration depths (FIG. 11D). Only brain tissue (from a cow brain sample) shows stronger scattering.

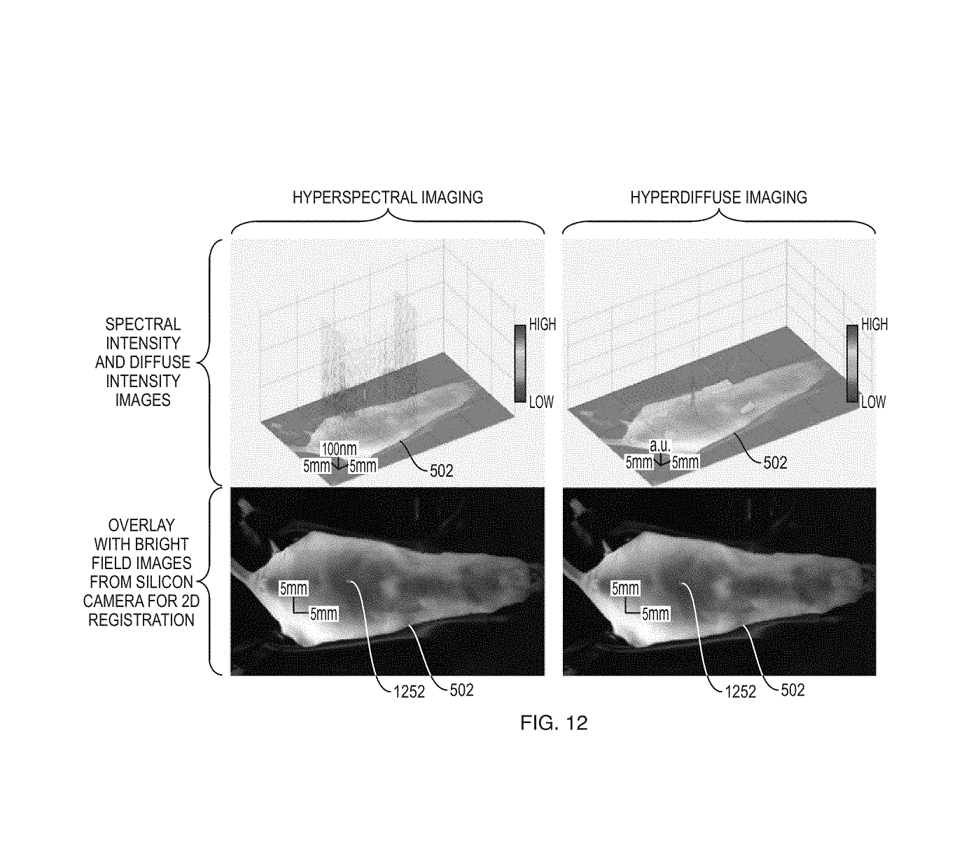

FIG. 12 illustrates spectral intensity (SI), diffuse intensity (DI), and bright-field image overlays for hyperspectral and hyperdiffuse imaging in the NIR-II optical imaging range, as used to track a small particle 1252 under a mouse target 502. The left column shows Hyperspectral imaging, while the right column shows hyperdiffuse imaging. The top panel shows Spectral Intensity (SI) and Diffuse Intensity (DI) images of a 1 mm UCNP, placed under the mouse subject 502, imaged over a 2 cm×1 cm area of scan. The bottom panel shows an overlay with bright field images captured using a silicon camera, to give a 2-D image registration.

Performing diffuse imaging at different prior-defined spectral bands result in maximum signal-to-noise ratio and penetration depth. Further, different diffuse scattering property at different spectral bands can be used as an indicator for tissue type.

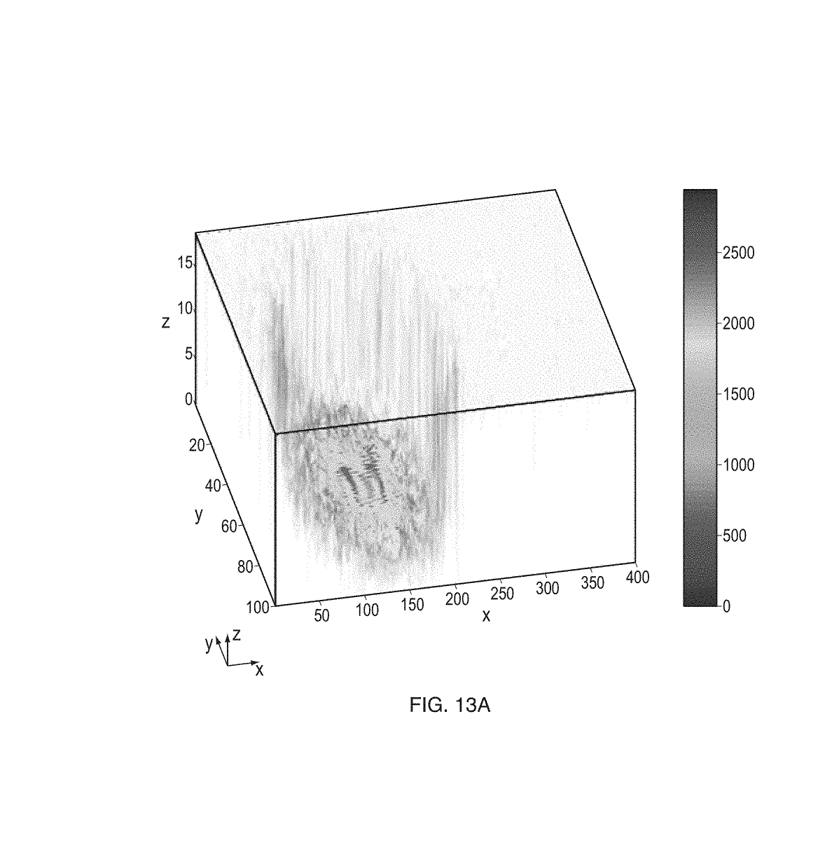

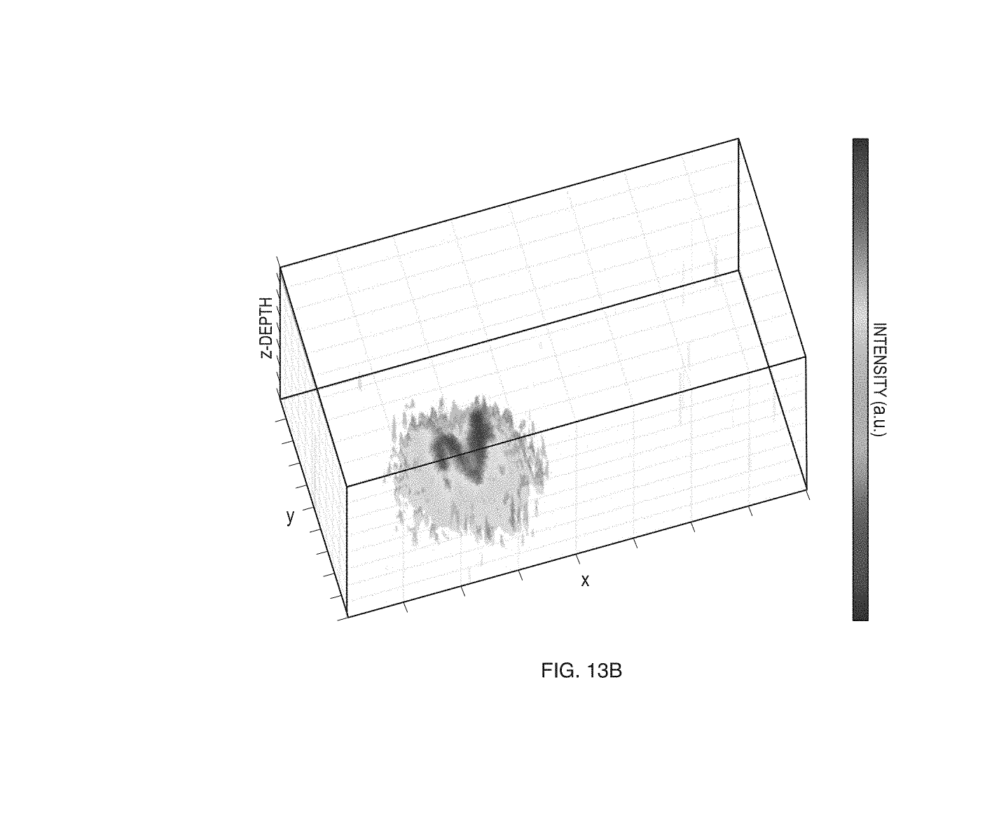

FIGS. 13A-13B show HDI imaging performed with a set of optical filters selected for allowing bandpass of the “M” in the “MIT”-shaped feature. The total scan size is 4 cm×1 cm on the XY axes respectively. FIG. 13A shows a 3-D overlay of the diffuse intensity image shown in FIG. 10B, with the scattering radius obtained from the diffuse width image shown in FIG. 10C. The overlay can be used for Z-depth estimation from analysis of HDC data. This is a demonstration of the 3-D fluorescent imaging obtained by combining DI with DW images. In conjunction with a surface plot obtained from the 3-D scanner, this can further be used for 3-D image registration.

FIG. 13B shows and XY planar projection of the image in FIG. 13A, constituting a 2-D fluorescent image. In conjunction with a photograph taken with the silicon camera, this can further be used for 2-D image registration. Conversely, from this plot, given a fluorescent spectrum of known intensity distribution, from the scattering radius it is possible to deduce the z-depth at the location of the fluorescent probe. Thus, based on diffuse width images corresponding to respective spectral band components, depth information for features in the inter-band image can be provided. It should be noted that depth is depth inside a surface of a target object, such as the “M” feature or a person's skin, for example, where the target object is represented in the inter-band image.

FIGS. 14A-14E illustrate various aspects of analysis of crude oil using an embodiment method and device. In particular, FIG. 14A is an illustration of a device, similar to the device illustrated in FIGS. 5A-5D, that can be used to obtain images in both HSI and HDI modes. The device in FIG. 14A is modified to accommodate a cuvette 1488 holding various samples of crude oil.

FIG. 14B is a graph illustrating know optical densities of crude oil and accompanying impurity components. With this information, impurities in crude oil can be detected by quantitatively and qualitatively.

FIGS. 14C-14D illustrate hyperspectral images obtained using a sample of light yellow crude oil. In FIG. 14C, the imaging was made with the target at a 0.5 cm depth. In FIG. 14D, the imaging was made with the target at a 5 cm depth. Based on the displayed results, it is estimated that imaging could be done with embodiment methods at depths up to 20 cm.

FIG. 14E illustrates hyperspectral images obtained using a dark black sample of crude oil, with the target at a depth of 2 cm. A limitation on depth in this case is laser power. A pulsed, high power laser can be used alternatively to improve the measured results and the possible range of imaging depths.

### FURTHER EXAMPLES OF TISSUE PENETRATION AND DEPTH MEASUREMENT AND 3D RECONSTRUCTION

### Effects of Thickness and Type of Tissues on Tissue Penetration

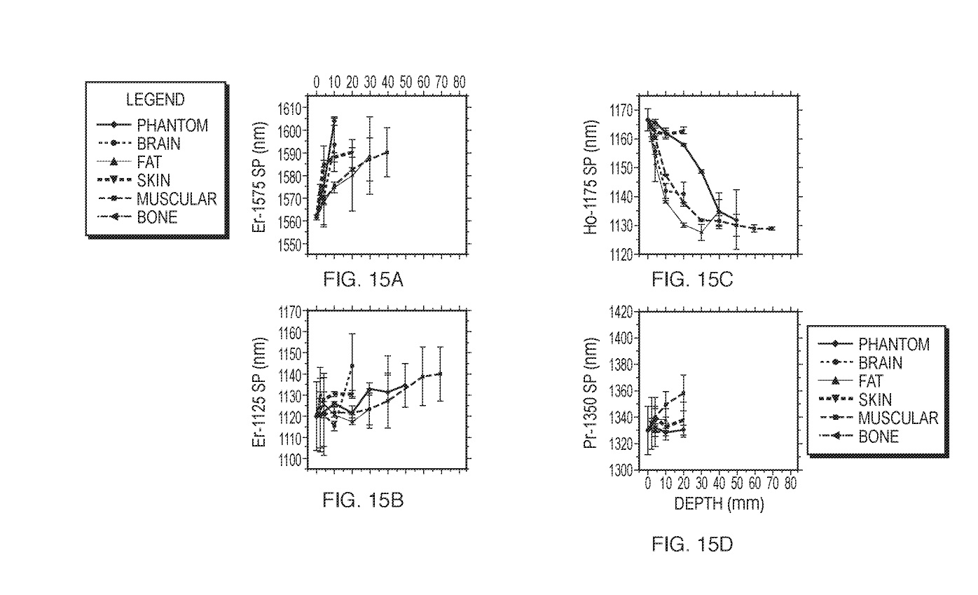

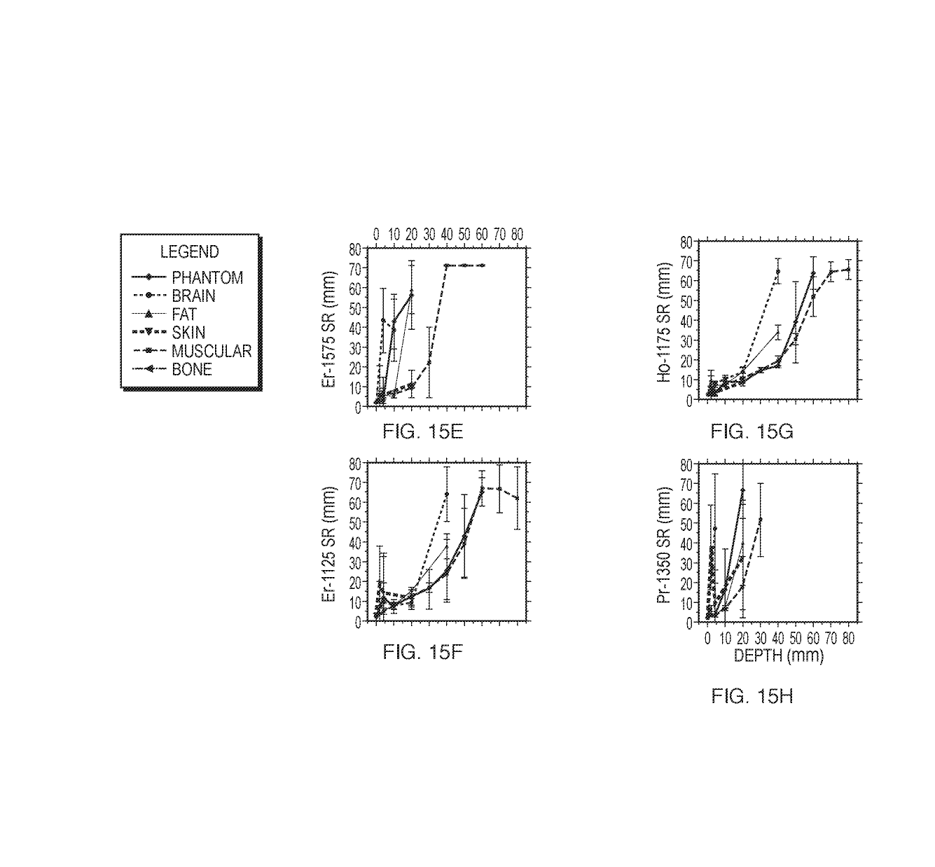

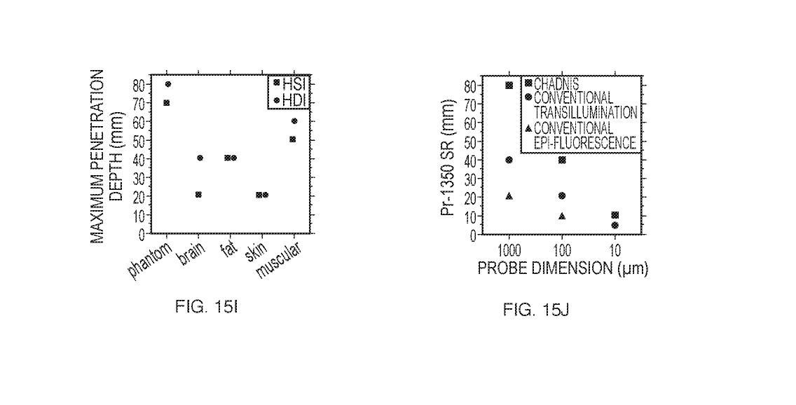

In view of the PCA, HSC, and HDC tools described hereinabove, together with the unique spectral and scattering analyses also described, the effects of tissue thickness and tissue type on the probe signal penetration were further studied. In particular, four distinct rare-earth emission peaks (Er-1575, Er-1125, Ho-1175, and Pr-1350) used, corresponding to measurements illustrated in FIGS. 15A-15H.

Similar to pixel-wise analysis of HSC, pixel-wise analysis of HDC results in Diffuse Intensity (DI) and Scattering Radius (SR) information for each pixel, denoted as DL and SRᵢ, respectively. It is noted that the DI is achieved by summarizing information of all scattering distances of HDC. For SR, similar to SP and SW, to accelerate the calculation, peak fitting is not used. Instead, the distance to 50% of maximum intensity is used as SR. The SRᵢ for each pixel are given by:

SR_{i=α-δ}(x,y)=Scattering Radiusᵣ(HDC_{i=α-δ}(x,y,r))

FIGS. 15A-15J are a series of graphs illustrating measured effects of thickness and tissue type on the spectral and scattering properties identified by DOLPHIN. Measurements for six types of tissues are presented, including breast-mimic phantom, brain, fat, skin, muscular, and bone tissues. Depending on the tissue type, tissue thickness varies from 2 mm to 80 mm.

FIGS. 15A-15D show SP measurements using the four distinct emission peaks, while FIGS. 15E-15H show SR measurements for the same four distinct NIR emissions. In particular, Er-1575 measurements are shown in FIGS. 15A and 15E); Er-1125 measurements are shown in FIGS. 15B and 15F; Ho-1175 measurements are shown in FIGS. 15C and 15G; and Pr-1350 measurements are illustrated in FIGS. 15D and 15H. Data in FIGS. 15A-15H are shown as mean±standard deviation (s.d.) for n≧10 samples (pixels used for calculation) at each depth, tissue type and probe condition. FIGS. 15A-H share the same legend as FIG. 15D.

FIG. 15I shows the maximum penetration depths achieved by HSI and HDI for the various types of tissues. FIG. 15K shows the maximum penetration depths through breast-mimic phantom achieved by DOLPHIN, as well as by conventional NIR-II imaging in transillumination and epi-fluorescence modes. For FIGS. 15I-J, the maximum penetration depths are achieved when at least one of the “M”, “I”, and “T” letters can be identified. For FIG. 15J, each probe dimension represents an estimation of the actual size of the probe, and the actual dimension is at most 2 times larger than the indicated size.

The tissues studied include breast-mimic phantom, as well as animal fat, skin, brain, muscle, and bone. The animal tissues were all obtained from anatomical parts of a cow from a slaughterhouse. For Er-1575 and Ho-1175, SP shows monotonic increase and decrease, respectively, as the penetration depths increase, as illustrated by FIGS. 15A and 15C. This change corresponds to the relation between the effective attenuation coefficient (μ_{eff}), mainly associated with various small molecules (e.g., water and fat), and the emission intensity of fluorescence probes as functions of wavelength. When the tissue absorbs stronger on one side of the emission peak, the peak position of transmitted spectra shift to the opposite side.

Various tissue types contain different composition of small molecules, resulting in different SP changes. For instance, at similar penetration depth, muscle, skin, and brain tissues show stronger SP shift for Er-1575 due to higher water content, while fat and brain tissues show stronger SP shift for Ho-1175 due to higher fat content. In contrast, for Er-1125 and Pr-1350, the change of SP results from the overlap of probe fluorescence and tissue autofluorescence (FIGS. 15B, 15D), considering that the autofluorescence contributes more for deeper penetration.

While SPs show systematic changes, SWs show less-regular tendencies with depths or types of tissue. Deeper penetration generally results in lower SNR, thus resulting in a broader peak, and the changes in SWs relate largely to the SNR of each measurement. For HDI, it was observed that SR increases as a function of penetration depth as expected (FIGS. 15E-15H). While most types of tissue exhibit similar diffuse scattering properties at comparable penetration depths, muscle and brain tissues scatter more strongly than other types. In summary, both SP from HSI and SR from HDI show a certain degree of penetration thickness and tissue type dependence, demonstrating an empirical means to identify signal depth and compositional variations in the environment surrounding the emitting probes.

Besides SP shifts of HSI, relative band intensity (i.e. comparing SI of different spectral bands) changes also relate to tissue absorption at different wavelengths. For example, emission at δ-band is stronger than β-band of Er-NP, while most tissues absorb more in the 6-band due to strong water absorption. As a result, Er-NP emission in δ-band shows stronger signals from phantom penetration of up to 20 mm, while the emission in β-band has stronger signals for more than 30 mm penetration. This observation appears not to have been reported or applied in existing methods, which have all employed the emission of Er-NP in δ-band. It is possible that the high level of autofluorescence signal in conventional epi-fluorescence imaging prevents effective imaging in the β-band, owning to small spectral separation from the excitation. Instead, the transillumination HSI in the NIR leads to the discovery of a previously unexplored imaging condition for deeper penetration, and consequently, we have demonstrated imaging with penetration up to 70 mm of breast-mimic phantom. Similarly, this imaging condition of using either Er-NP or Ho-NP emitting at 1125 nm or 1175 nm has been applied to both HSI and HDI for a variety of tissues to achieve maximum depths of penetration (FIG. 15I).

Notably, for all major types of tissues, the maximum depths of penetration are more than 4 cm, particularly 8 cm and 6 cm for breast-mimic phantom and muscular tissue from HDI, and 7 cm and 5 cm for breast-mimic phantom and muscular tissue from HSI (FIG. 15I). Particularly, penetrating through 8 cm of phantom is close to the theoretically-predicted limit of detection through 10 cm of biological tissue. Nonetheless, we consider that the penetration capability of NIR imaging by DOLPHIN could be further advanced by optimized fluorescence probes, imaging optics, as well as processing methods. In addition, compared to the conventional NIR-II imaging in both transillumination and epi-fluorescence modes, DOLPHIN greatly enhances the maximum penetration depths (see FIG. 15J), and demonstrates detection of probes of 10 or 100 μm through 1 or 4 cm of breast-mimic phantom. DOLPHIN can, thus, be used as a platform for detection of cellular-sized features through deep biological tissues, and for tracking physico-chemical phenomena of interest through either endogenously expressed fluorescent reporters, exogenously introduced targeted fluorescent contrast agents, or inherent heterogeneities in the specimen. Therefore, embodiments provide imaging capability at various hierarchical scales of interest, from the cellular level to whole animal.

### Depth and Effective Attenuation Coefficient

While tabulated results from extensive measurements of tissue penetration studies allow empirically determining the depth of fluorescence signal for HSI and HDI in certain special cases (e.g., cylindrical symmetry of tissue inspected is required for HDI analysis), a more general approach to determining signal depth and optical properties of the surrounding environment has additional benefits. Disclosed herein are further methods of determining depth and surrounding environment of fluorescence signals and reconstructing 3-D images using DOLPHIN.

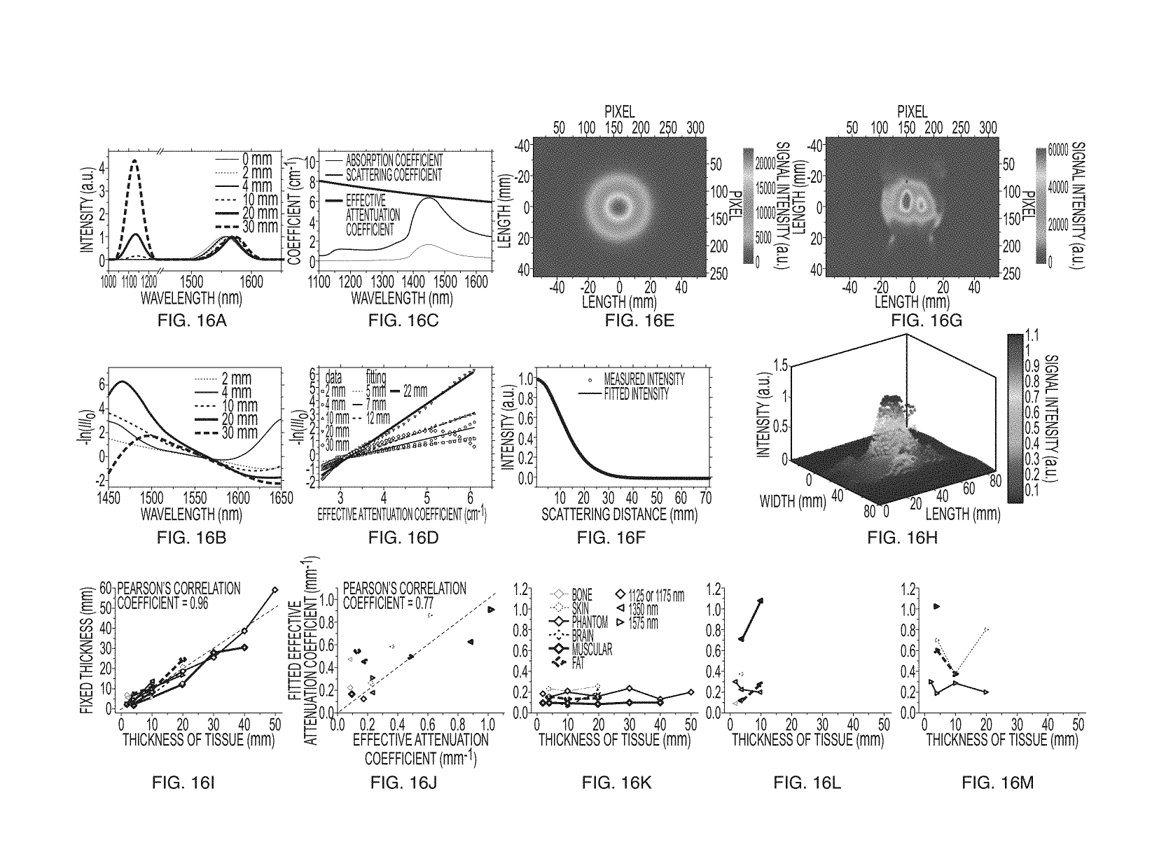

FIGS. 16A-16M are graphs illustrating derivation of depth of signal and effective attenuation coefficient of tissues from fitting the results of tissue or animal penetration by DOLPHIN. In particular, FIGS. 16A-16B illustrate the emission spectra normalized at Er-1575 peak (FIG. 16A) and -ln(I/I₀)) (FIG. 16B, where I and I₀ are the transmitted emission intensity through tissue and the intrinsic emission intensity) of Er-NP measured from HSI, penetrating through 0-30 mm of breast-mimic phantom. FIG. 16C illustrates the estimated absorption coefficient, scattering coefficient, and effective attenuation coefficient of breast tissue. FIG. 16D illustrates fitting of tissue depth using Beer's law and the data presented in FIGS. 16B-C.

FIG. 16E shows the 2-D scattering profile detected from photon-exiting plane for a 2-cm-thick breast-mimic phantom, as measured by HDI. The scattering profile in FIG. 16E shows cylindrical symmetry. FIG. 16F illustrates the corresponding 1-D scattering profile as a function of scattering distance, with data points in black. The fitted results in FIG. 16F use depth and effective attenuation coefficient as fitting parameters (red line), which is in excellent agreement with the measured data.

FIG. 16G shows a representative 2-D scattering profile measured by HDI for a whole mouse. The scattering profile in FIG. 16G shows no cylindrical symmetry. Similar to the tissue penetration results, the fluorescence probe of Er-NP is placed directly underneath the mouse at the location with maximum height. FIG. 16H illustrates the measured (shown as 3-D scattered points) and fitted results (shown as solid surface profile) for the mouse imaging using the generalized fitting equation. In FIG. 16H, depth and effective attenuation coefficient are used as the fitting parameters.

FIGS. 16I and 16J show the fitted thicknesses (FIG. 16I) and effective attenuation coefficients (FIG. 16J) compared to the actual thicknesses of the tissues and the estimated effective attenuation coefficients. The black dashed lines in FIGS. 16I and 16J represent equivalency between fitted and actual values.

FIGS. 16K-16M illustrate the fitted effective attenuation coefficients for various tissues and thicknesses at wavelengths of 1125 nm or 1175 nm (FIG. 16K), 1350 nm (FIG. 16L), and 1575 nm (FIG. 16M). FIGS. 16I-16M share the same legends shown in FIG. 16K.

For HSI, in order to calculate the depth of the fluorescence signal at a certain spatial position (x,y), Beer's law can be applied, ln

$$\frac{I ( λ )}{{I}_{0} ( λ )} = - d \cdot {μ}_{eff} ( λ ) + constant ,$$

where I₀(λ), I(λ), and ∥_{eff}(λ), respectively, are the intrinsic fluorescence intensity of the probe at zero depth of penetration, measured fluorescence intensity through tissue, and the effective attenuation coefficient of tissue as functions of wavelength. The signal depth d can be obtained by linearly fitting

$$ln \frac{I ( λ )}{{I}_{0} ( λ )}$$

with respect to μ_{eff}(λ).

FIGS. 16A-16C show the measured emission spectra of Er-1575 band from HSI and estimated μ_{eff}(λ) of a breast-mimic phantom. The fitted results for tissue penetration up to 20 mm match well to the actual depths (FIG. 16D), indicating the effectiveness of calculating depth from HSI. It is noted that calculating signal depth from HSI would be particularly effective in the case that, sufficient level of emission signals can be achieved for a range of wavelengths with different μ_{eff}, e.g. 1100-1200 nm, 1500-1600 nm.

For calculating depth and μ_{eff} from HDC, the case of cylindrical symmetry was first considered. In particular, it was considered, for this purpose, that the scattering profile possesses cylindrical symmetry, I(r), for regularly shaped and uniform tissues (FIGS. 16E-16F). Assuming that the emitted light travels from depth d as a spherical wave in a homogeneous optical medium with μ_{eff}, the equation:

$$I ( r ) \cdot ( {r}^{2} + {d}^{2} ) \cdot {e}^{{u}_{eff} \cdot \sqrt{{r}^{2} + {d}^{2}}} = constant$$

can be used to fit both depth d and μ_{eff}. The fitted results match well to the actual depth as well as estimated μ_{eff} for various tissues (FIGS. 16F and 16I-16M).

Further, for a general case without cylindrically symmetric scattering profile I(a, b) due to irregular shape of the tissue or animal (FIG. 16G), a similar relation can be used to calculate the probe depth by (1) assuming the tissue is a homogeneous optical medium and (2) using the height/surface profile of the subject obtained by a 3-D scanner. For this case, the fitting equation changes to:

$$I ( a , b ) \cdot ( {r}^{2} + {d}^{2} ) \cdot {e}^{{u}_{eff} \cdot \sqrt{{r}^{2} + {d}^{2}}} = constant ,$$

where r²=(a−a₀)²+(b−b₀)², h=d−z(a₀, b₀)+z(a, b). The parameters a and b are the in-plane spatial coordinates, and z is the height at each location of (a, b). The parameters a₀ and b₀ are the center location of the incident beam.

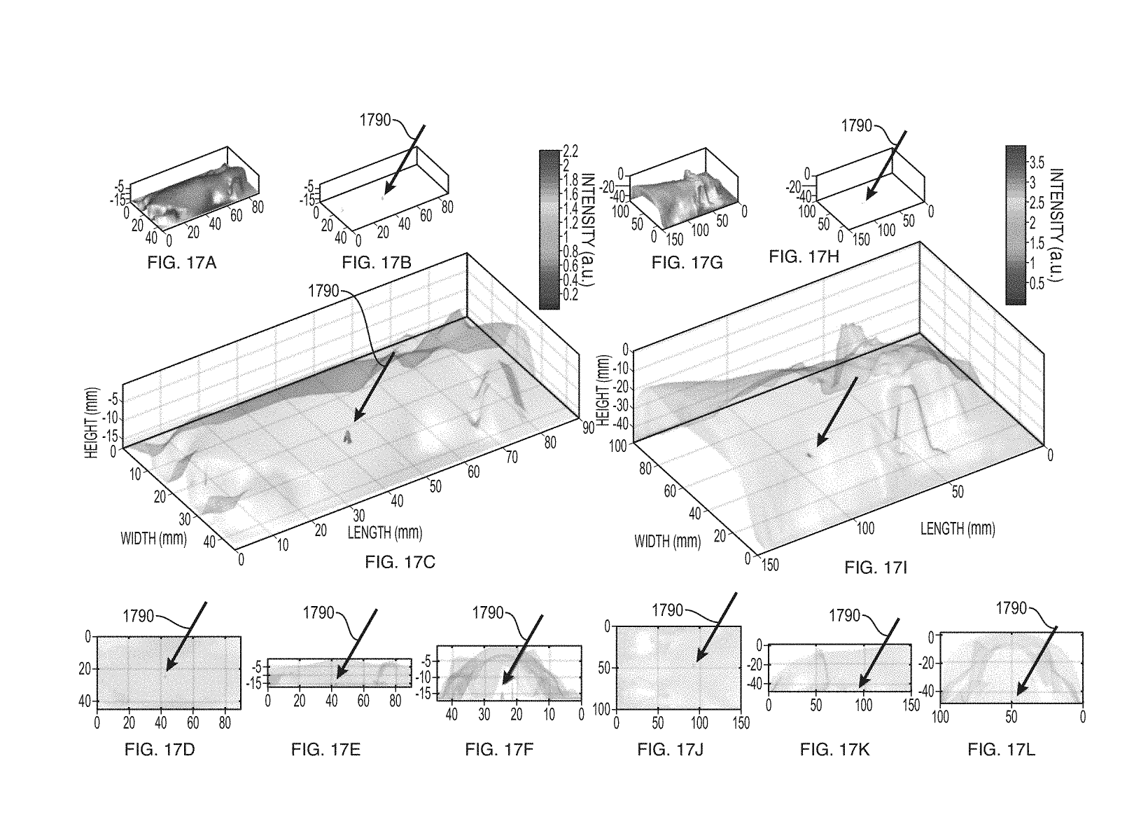

Combining the fluorescence signal I(a, b) and the 3-D scanned height profile z(a, b), both depth d and μ_{eff} can be fitted (FIG. 16H). The fitted results for depth d are in agreement with the actual value (which can be seen in the 3-D reconstruction described hereinafter in connection with FIGS. 17A-17F), though the residual of the fitness is larger than the cylindrical homogeneous case mainly due to the simplifying assumption of homogeneity for a heterogeneous subject, in particular the animal.

Overall, it is demonstrated that signal depth can be derived from both HSI and HDI. While HSI has the advantage of identifying autofluorescence, HDI offers more accurate results of fitted depth for a large variety of tissues without the knowledge of the type of tissue. Additionally, HDI predicts μ_{eff} sufficiently close to the estimated values with the scattering profiles as the only information, which exhibits opportunity to identify and distinguish different types of tissues.

### Fluorescence 3-D Reconstruction of Animal Imaging

FIGS. 17A-17L are constructed graphical images illustrating fluorescence 3-D reconstruction of animal imaging. In particular, FIGS. 17A-17F show fluorescence 3-D reconstruction of 100-μm-size Er-NP detected through a whole-mouse approximately 2 cm thick. FIGS. 17G-L illustrate fluorescence 3-D reconstruction of 1-mm-size Er-NP detected through a rat approximately 4 cm thick.

Further details for these figures include the following. FIGS. 17A and 17G are surface profiles of the animals measured by a 3-D scanner (NextEngine HD®). The 3-D scanner generates a point cloud of the top surface of the scanned object (here, an animal), and the point cloud is subsequently stitched together to form the 3D image. FIGS. 17B and 17H are reconstructed height profiles of the fluorescence signals measured from HDI, i.e., 3-D fluorescence images. FIGS. 17C and 17I are overlays of the 3-D bright-field and fluorescence images. FIGS. 17D-17F and 17J-17L are top views along the z-axis (FIGS. 17D and 17J), side views along the y-axis (FIGS. 17E and 17K), and side views along the x-axis (FIGS. 17F and 17L, respectively, of the 3-D overlay images. The arrows 1790 in FIGS. 17B-17F and 17H-17L point to the locations of the identified fluorescence probes.

By combining the 2-D fluorescence contrast images from DOLPHIN with the calculated height profiles of the fluorescence signal, a 3-D fluorescence signal reconstruction can be achieved. In the illustrated examples of determining the penetration using whole animals, it was observed that 100 μm size Er-NP can be detected through the whole body of a mouse (approximately 2 cm thick, FIGS. 17A-17F), and 1 mm size Er-NP can be detected through the whole body of a rat (approximately 4 cm thick, FIGS. 17G-17L). The reconstructed 3-D fluorescence images and the side views clearly show the deep positions of the fluorescence probes of small sizes.

Of further significance, the 100 μm size Er-NP is considered close to cellular size of animals and human, which is in the range of 10-100 μm. Thus, this is a demonstration of using DOLPHIN to perform cellular sized feature detection through deep tissue or whole animal. Disclosed methods and systems, thus, can enhance the application of fluorescence imaging significantly past what has been previously achieved. In addition, unlike the tomographic methods for reconstructing 3-D fluorescence images by collecting spatial information from multiple imaging planes, DOLPHIN can collect spatial information and spectral or scattering information from one imaging plane, and the height profile of the fluorescence signal can be achieved by analyses of the spectral or scattering information.

### Label-Free DOLPHIN-Based Imaging

In addition to the aforementioned sources of endogenous and exogenous contrast, embodiment methods and systems also extend to performing “label-free” imaging, without relying on either endogenous or exogenous contrast sources. DOLPHIN, for example can be designed to perform label-free HSI/HDI, without the use of either kind of contrast agent. This can be enabled, for example, by the use of alternative image contrast mechanisms that are inherent to the specimen being imaged. The sources of these image contrast mechanisms can include numerous heterogeneities (inherent heterogeneities), such as: (a) tissue autofluorescence caused by inelastic Raman scattering due to lipids, as described hereinabove, (b) compositional differences arising due to varying content of fat, water and other scatterers such as blood in tissues, (c) density differences, which are related to tissue composition, such as bone being denser than fat or muscle, and (d) differences in oxygenation (hypoxia) or pH (acidic) in tumor tissue relative to healthy tissue. Inherent heterogeneities can also be present and exploited for imaging in non-tissue media, such as the petroleum-based media described in connection with FIG. 14A.

Combined with a tunable-wavelength (ranging from 690-1,040 nm) laser equipped on a DOLPHIN imaging system (as illustrated in FIGS. 5A-5D, e.g.) for scanning through a continuum of incident (excitation) wavelengths, it is possible to adapt the image-processing methods described herein to be able to detect such fine micro-scale compositional variations by resolving their unique spectral signatures, which can be applied for label-free early diagnostics. This can be especially useful for diagnostic applications in which endogenous contrast agents are not usually expressed (such as the human body), or where there is no a priori knowledge of the presence of a particular kind of disease (such as during regular annual physical exams) motivating the introduction of a cocktail of exogenous contrast agents into the body.

FIGS. 18A-18E are graphical images illustrating a “label-free” scan of a healthy, non-diseased mouse 1892. In particular, FIGS. 18A-B show the mouse lying in prone (FIG. 18A) and supine (FIG. 18B) positions, and FIG. 18C shows organs removed after euthanizing the animal, including a bladder 1894a, spleen 1894b, heart 1894c, spine 1894d, ovary 1894e, pancreas 1894f, lung 1894g, liver 1894h, kidney 1894i, intestine 1894j, sternum 1894k, and stomach 18941. There was no contrast agent used, either natively expressed in the animal, or injected externally. The various organs 1894a-1 are distinguishable both in the whole body and upon excision.

FIG. 18E is a graphical plot showing the grid used for raster scanning of the animal, with the position of the subject mouse 1892 outlined for clarity. FIG. 18D is an inset of FIG. 18E, showing one example of the gridpoint data, with the horizontal and vertical (X- and Y-axes, respectively) corresponding to excitation and emission, respectively. Note that the horizontal (X)-axis runs from 690-1,040 nm (corresponding to the wavelength-tunable laser described hereinabove), while the vertical (Y)-axis ranges from 850-1,650 nm.

Thus, embodiment methods and systems can include obtaining hyperspectral image data without exogenous or endogenous labels. Spectral band components corresponding to mutually distinct sources of image contrast can correspond result from heterogeneities in a subject, such as the mouse 1892, represented in the hyperspectral image data, such as the data illustrated in FIG. 18E. Furthermore, in the event of a positive detection of a given feature using a label-free technique, it is also possible to follow up with use of an actively-targeted, exogenous contrast agent for higher signal-to-background and improved sensitivity.

The teachings of any patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method comprising:
identifying a plurality of wavelength spectral band components in hyperspectral image data, the spectral band components corresponding to mutually distinct sources of image contrast;
calculating respective intensity images corresponding to each respective spectral band component;
combining the respective intensity images to form inter-band images based on the respective, mutually distinct sources of image contrast for each spectral band component; and
producing a 2D image of enhanced contrast using selection of one or more of the inter-band images that are of greater contrast than one or more others of the inter-band images.

2. The method of claim 1, wherein calculating the respective intensity images includes performing an intra-band pixel-wise analysis of one or more of the spectral band components.

3. The method of claim 1, wherein combining the respective intensity images to form an inter-band image includes performing an inter-band pixel-wise analysis by dividing individual pixel values of one of the intensity images by corresponding individual pixel values of another of the intensity images.

4. The method of claim 3, wherein performing the inter-band pixel-wise analysis further includes dividing individual pixel values of more than one of the intensity images by corresponding individual pixel values of others of the respective intensity images, respectively, to form the plurality of inter-band images.

5. The method of claim 1, wherein the respective intensity images are respective diffuse intensity images, the method further comprising obtaining hyperdiffuse image data for each spectral band component in the hyperspectral image data.

6. The method of claim 5, further including enhancing contrast for a respective diffuse intensity image based on a maximum radial distance max r calculated from a plurality of radial distances r, where r is a distance between a given pixel of the respective hyperdiffuse image data and a center pixel corresponding to an incident beam location identified in the respective hyperdiffuse image data.

7. The method of claim 5, further including enhancing contrast for each respective diffuse intensity image based on a median r, where r is a distance between a given pixel of the respective hyperdiffuse image data and a center pixel corresponding to an incident beam location identified in the respective hyperdiffuse image data.

8. The method of claim 5, further including enhancing contrast for each respective diffuse intensity image based on a principal component analysis (PCA) score r(PCA), where r is a distance between a given pixel of the respective hyperdiffuse image data and a center pixel corresponding to an incident beam location identified in the respective hyperdiffuse image data.

9. The method of claim 5, further including calculating respective diffuse width images corresponding to respective spectral band components to provide depth information for features in the inter-band image, the depth being depth inside a surface of a target represented in the inter-band image.

10-13. (canceled)

14. The method of claim 1, wherein the respective intensity images are respective spectral intensity images, and wherein calculating the respective spectral intensity images includes using the hyperspectral image data as source data.

15. The method of claim 14, wherein calculating each respective spectral intensity image includes calculating based on a wavelength of maximum intensity identified in the respective spectral band.

16. The method of claim 14, wherein calculating each respective spectral intensity image further includes calculating based on a wavelength of median intensity identified in the respective spectral band.

17. The method of claim 14, wherein calculating each respective spectral intensity image further includes calculating based on a wavelength of highest principal component analysis score determined for the respective spectral band.

18. The method of claim 1, further including ascertaining the mutually distinct sources of image contrast for the respective spectral band components based on spectral position images or spectral width images for the respective spectral bands.

19. The method of claim 1, wherein the target medium is a three-dimensional (3-D) target medium, the method further comprising determining lateral, two-dimensional (2-D) location of one or more features in the target medium and depth of the one or more features from a surface of the target medium.

20-23. (canceled)

24. The method of claim 19, wherein determining depth includes determining a depth in a range from 0 cm to about 2 cm.

25. The method of claim 19, wherein determining depth includes determining a depth in a range from about 2 cm to about 3.2 cm.

26. The method of claim 19, wherein determining depth includes determining a depth in a range of about 3.2 cm to about 5 cm.

27. The method of claim 19, wherein determining depth includes determining a depth in a range of about 5 cm to about 9 cm.

28-30. (canceled)

31. The method of claim 1, further including obtaining the hyperspectral image data by illuminating a target medium with incident light.

32. The method of claim 31, wherein illuminating the target medium with the incident light includes using a light source having a wavelength between about 750 nm and about 1600 nm.

33. The method of claim 31, wherein illuminating the target medium with the incident light includes using a light source having a wavelength between about 750 nm and about 1100 nm.

34. The method of claim 31, wherein obtaining the hyperspectral image data includes using a forward imaging mode with the target medium positioned in an optical path between a light source illuminating the target medium and a detector array configured to detect a hyperspectral image from which the hyperspectral image data are derived, the inter-band image being an image of the target medium.

35. The method of claim 31, wherein obtaining the hyperspectral image data includes using a reflectance imaging mode, with a detector array positioned to substantially avoid detection of light from a light source illuminating the target medium, wherein the detector array is configured to detect a hyperspectral image from which the hyperspectral image data are derived, the inter-band image being an image of the target medium.

36. The method of claim 31, wherein obtaining the hyperspectral image data includes using an angular imaging mode, with the target medium being in an optical path between a light source illuminating the target medium, and a detector array configured to detect a hyperspectral image from which the hyperspectral image data are derived, the detector array positioned at an angle with respect to the illuminating light source in a range of about 0° to about 180°, from which the hyperspectral image data are derived, the inter-band image being an image of the target medium.

37. (canceled)

38. The method of claim 31, wherein illuminating the target medium with the incident light includes illuminating a probe introduced to the target medium, and wherein identifying the plurality of spectral band components includes identifying a spectral band component corresponding to emission from the probe.

39-41. (canceled)

42. The method of claim 1, wherein combining the respective intensity images to form the inter-band image includes forming an image of a cell, tissue, organ, tumor, or whole body.

43. The method of claim 1, wherein combining to form the inter-band image includes forming an image of a fossil fuel.

44. The method of claim 1, wherein combining to form the inter-band image includes forming an image with a resolution at a single-cell level.

45-48. (canceled)

49. The method of claim 1, further comprising obtaining the hyperspectral image data without exogenous or endogenous labels, and wherein the spectral band components correspond to mutually distinct sources of image contrast that result from heterogeneities in a subject represented in the hyperspectral image data or in hyperdiffuse image data.

50. (canceled)

51. An imaging system comprising:
a detector configured to acquire hyperspectral image data for a target; and
one or more processors configured to identify a plurality of wavelength spectral band components in the hyperspectral image data, the spectral band components corresponding to mutually distinct sources of image contrast, the one or more processors being further configured to calculate respective intensity images corresponding to each respective spectral band component and to combine the respective intensity images to form inter-band images based on the respective, mutually distinct sources of image contrast for each spectral band component the one or more processors being still further configured to produce a 2D image of enhanced contrast using selection of one or more of the inter-band images that are of greater contrast than one or more others of the inter-band images.

52. A method comprising:
identifying a plurality of wavelength spectral band components in a hyperspectral image of a target, the spectral band components corresponding to mutually distinct sources of image contrast;
transforming each respective spectral band component to obtain a spectral position image and a spectral width image corresponding to each respective spectral band component; and
producing a 3D image of one or more features inside a surface of the target based on the spectral position images and the spectral width images.

53. The method of claim 52, wherein identifying the plurality of wavelength spectral band components includes identifying optical spectral band components.

54. The method of claim 31, wherein illuminating the target medium with the incident light includes using a light source having a wavelength between about 900 nm and about 1400 nm.
